(19) United States
(12) Patent Application Publication
(10) Pub. No.: US 2025/0290036 A1
(43) Pub. Date: Sep. 18, 2025

(54) METHODS FOR DECELLULARIZING HUMAN OMENTUM AND PRODUCTS GENERATED THEREFROM

(71) Applicants: MATRICELF LTD., Ness Ziona (IL); RAMOT AT TEL-AVIV UNIVERSITY LTD., Tel-Aviv (IL)

(72) Inventors: Tamar HAREL ADAR, Tel-Aviv (IL); Tal DVIR, LeHavim (IL); Alex SAAR, Ramat Gan (IL); Shirly TZELIK, Rishon LeZion (IL); Ron GOLDNER, Rehovot (IL); Tammy BEN MORDECHAI, Rishon LeZion (IL); Ilana GROSS CARMEL, Givat Shmuel (IL); Tal BEN NERIAH, Herzliya (IL)

(73) Assignees: MATRICELF LTD., Ness Ziona (IL); RAMOT AT TEL-AVIV UNIVERSITY LTD., Tel-Aviv (IL)

(21) Appl. No.: 19/221,545

(22) Filed: May 29, 2025

Related U.S. Application Data
(63) Continuation of application No. PCT/IL2023/051227, filed on Nov. 29, 2023.
(60) Provisional application No. 63/428,462, filed on Nov. 29, 2022.

(51) Int. Cl. C12N 5/0793; A61K 35/30; C12N 5/071; C12N 5/074; C12N 9/64; C12N 9/76; C12N 9/94

(52) U.S. Cl. CPC C12N 5/0619; A61K 35/30; C12N 5/0696; C12N 5/0697; C12N 9/6427; C12N 9/6481; C12N 9/94; C12Y 304/21002; C12N 2506/45; C12N 2513/00

(57) ABSTRACT

A composition is disclosed which comprises solubilized decellularized omentum. Uses thereof and methods of generating same are also disclosed.

### CROSS-REFERENCE TO RELATED APPLICATIONS

This is a Continuation of International Patent Application No. PCT/IL2023/051227, filed on Nov. 29, 2023, which claims the benefit of priority of U.S. Provisional Patent Application No. 63/428,462, filed on Nov. 29, 2022. The contents of the foregoing patent applications are incorporated herein by reference in their entirety.

### FIELD AND BACKGROUND OF THE INVENTION

The present invention, in some embodiments thereof, relates to an improved method for decellularizing omentum, decellularized omentum matrices for tissue engineering and gels comprising decellularized omentum.

Tissue engineering is a multidisciplinary field that combines cells, biomaterials, and engineering tools, with aim to restore damaged tissues, generate functional new tissues and even create full functional organs. The components are integrated and undergo specific processes to design the desired function of the engineered tissue. To ensure beneficial function, each one of the components must be carefully selected.

The cellular component of the engineered tissue is a crucial component, dictating the function as well as the safety of the end product. The safety of the incorporated cells is mainly affected by their source; as cells originating from donors that are not perfectly matched to the recipient, might generate an immune response that could lead to rejection of the implanted cells. Therefore, immunological compatibility is highly desired, and attempts to identify a high degree of matching are pursued when allogeneic cells are being used. Researchers in the field of tissue engineering, throughout the world, have been addressing this challenging issue over the past years and have included mature cells, multipotent and pluripotent cells from various sources, each presenting specific benefits and drawbacks.

The second component of an engineered tissue is the biomaterial as an inductive template for tissue assembly. Over the years, this field has dramatically evolved to address the challenge of designing the ultimate material for this purpose in terms of safety and functionality. Synthetic polymers with a wide range of mechanical properties have been used to accommodate cells and support their growth. However, scaffolding materials that provide both physical support and, also actively participate in physiological processes, could be advantageous, and provide cues which may promote cell adhesion, migration and angiogenesis. Thus, biological motifs and bio-factors were incorporated into or on the backbone of polymeric matrices. For example, macroporous scaffolds were modified with RGD or YIGSR peptides to encourage cell adhesion, and VEGF was inserted into self-assembled fibers to promote vascularization. In other works, bio-factor-releasing systems were incorporated into scaffolds to control the release of growth factors and cytokines. However, as the research and development in the field have progressed, it was apparent that it is impossible to fully mimic the natural structure, composition and properties of the extracellular matrix (ECM) with synthetic or non-ECM materials.

Various animal-sourced tissues have been decellularized and used as a scaffold to engineer various types of tissues (Xuewei Zhang, 2022, Bioactive Materials, Volume 10, 2022, Pages 15-31, ISSN 2452-199X).

Decellularization is a process used for the separation of the extracellular matrix (ECM) from other tissue components such as fat, cells and cell debris. The outcome is a scaffolding biomaterial that may be used for various applications, including tissue engineering. Such ECM-based scaffolds support cell attachment, proliferation and, under specific conditions, may promote stem cell differentiation. In many cases, the ECM-based material can be further processed into an ECM-based hydrogel, which may be injected into desired locations in the body to promote tissue and organ regeneration.

Many patients worldwide were treated with animal-derived ECM-based products with promising results. However, the associated reported immune response raised a major concern. Despite the efforts to reduce the immunogenicity of these animal-derived biomaterials, traces of immunogenic epitopes still remained in the processed ECM (Badylak et al., Epub 2007 May 8. PMID: 17524477). This may be attributed to the presence of animal-specific antigens that humans lack, and to the differences in amino acid sequences of ECM proteins that animals and humans share. For example, up to 37% difference in collagen type IV sequence was observed between porcine and humans.

Additionally, simulation of the immune response in humans, when subjected to human and animal materials, revealed a significantly lower degree of T cell activation in response to the human materials. Furthermore, in vivo experiments using a probe for the production of reactive oxygen species (ROS) revealed significantly lower levels of inflammation in animals transplanted with autologous materials than allogeneic or xenogeneic materials.

Dvir, T., et al. (Proc Natl Acad Sci U S A 106, 14990-14995 (2009)) teach the utilization of the omentum to induce cell migration and blood vessel network formation in an implanted scaffold. These vascularized scaffolds were then re-implanted on the infarcted heart and completely attenuated its deterioration.

International Patent Application No. WO2009/085547 teaches the generation of decellularized omentum scaffolds for tissue engineering. International Patent Application No. WO2009/085547 does not teach use of the decellularized omentum scaffolds for cardiac engineering.

International Patent Application No. WO2014/207744 teaches the generation of decellularized omentum scaffolds for tissue engineering. International Patent Application No. WO2014/207744 does not teach conditions for decellularizing human omentum.

U.S. Patent Publication No. 20050013870 teaches a scaffold comprising decellularized extracellular matrix of a number of body tissues including omentum. The body tissues have been conditioned to produce a biological material such as a growth factor.

Porzionato et al. (Italian Journal of Anatomy and Embryology, Volume 116, 2011 and Eur J Histochem. 2013 Jan. 24; 57(1):e4. doi: 10.4081/ejh.2013.e4) teaches decellularized omentum.

Additional background art includes Gilbert et al., Biomaterials 27 (2006) 3675-3683and Flynn et al., Biomaterials 31 (2010), 4715-4724.

U.S. Patent Publication No. 20090163990 teaches methods of decellularizing omentum.

Soluble forms of decellularized extracellular matrix are known in the art as described in Acta Biomaterialia, Volume 9, Issue 8, August 2013, Pages 7865-7873 and Singelyn et al., J Am Coll Cardiol. Feb. 21, 2012; 59(8): 751-763.

### SUMMARY OF THE INVENTION

According to an aspect of the present invention, there is provided a method of decellularizing omentum comprising cutting the omentum into pieces having a surface area between 25-80 mm² and a volume of 13-70 mm³;
a. exposing the omentum to a hypotonic solution following step (a);
b. dehydrating the omentum following step (b);
c. extracting fat from the dehydrated omentum using polar
d. and non-polar solvents following step (c);
e. rehydrating the dehydrated omentum following step (d);
f. removing cell debris from the rehydrated omentum using an enzyme selected from the group consisting of TrypLE™ Select, TrypLE™ Express and Trypsin-EDTA following step (e); and
g. degrading nucleic acid from the rehydrated omentum following step (f) using endonuclease Benzonase® or Denarase®, thereby generating decellularized omentum.

According to another aspect of the present invention, there is provided a method of decellularizing omentum comprising:
cutting the omentum into pieces having a surface area between 25-150 mm² and a volume of 13-125 mm³;
exposing the omentum to a hypotonic solution following step (a); dehydrating the omentum following step (b);
extracting fat from the dehydrated omentum using polar and non-polar solvents following step (c);
rehydrating the dehydrated omentum following step (d);
removing cell debris from the rehydrated omentum using an enzyme selected from the group consisting of TrypLE™ Select, TrypLE™ Express and Trypsin-EDTA following step (e); and
degrading nucleic acid from the rehydrated omentum following step (f) using endonuclease Denarase®, thereby generating decellularized omentum.

According to embodiments of the invention, the non-polar extraction solvent is hexane.

According to embodiments of the invention, the polar extraction solvent is acetone or isopropanol.

According to embodiments of the invention, the polar extraction solvent is acetone and the non-polar extraction solvent is hexane.

According to embodiments of the invention, the dehydrating is effected using a dehydrating agent selected from the group consisting of methanol, ethanol, isopropanol, propanol and combinations thereof.

According to embodiments of the invention, the TrypLE™ Select or the TrypLE™ Express is contacted with the rehydrated omentum for at least one hour at 37° C.

According to embodiments of the invention, the method further comprises freezing the decellularized omentum following step (g) According to embodiments of the invention, the omentum is human omentum.

According to an aspect of the present invention, there is provided a composition of matter comprising decellularized omentum generated according to the method described herein.

According to another aspect of the present invention, there is provided a composition of matter comprising non-solubilized, decellularized omentum, wherein more than 40% of the total collagen content is collagen type I.

According to embodiments of the invention, the decellularized omentum comprises human omentum.

According to still another aspect of the present invention, there is provided a composition of matter comprising solubilized, decellularized human omentum, wherein more than 40% of the total collagen content is collagen type I, wherein less than 20% of the total protein content is the alpha I chain of type III collagen.

According to an aspect of the present invention, there is provided a scaffold fabricated from the composition of matter described herein.

According to an aspect of the present invention, there is provided a method of generating a cell-seeded scaffold comprising:
providing the scaffold described herein; and
seeding cells on the scaffold, thereby generating the cell-seeded scaffold.

According to an aspect of the present invention, there is provided an article of manufacture comprising a population of cells seeded on the scaffold described herein.

According to an aspect of the present invention, there is provided a composition of matter comprising solubilized decellularized omentum, having less than 100 μg sulfated Glycosaminoglycans (GAGs) per mL of hydrogel.

According to embodiments of the invention, the omentum is human omentum.

According to embodiments of the invention, the composition is in a liquid form.

According to embodiments of the invention, the composition has a gelation profile according to FIG. 6B, 6C or 6H.

According to embodiments of the invention, the composition has a gelation profile according to FIGS. 16B, 16C or 16D.

According to embodiments of the invention, the composition is in a gel form.

According to an aspect of the present invention, there is provided a method of generating a liquid composition of matter suitable for tissue generation, which, upon temperature activation, is capable of solidifying, the method comprising: decellularizing omentum to generate decellularized omentum; and solubilizing the decellularized omentum whilst in a wet state, thereby generating the composition.

According to embodiments of the invention, the solubilizing is effected using a protease enzyme.

According to embodiments of the invention, the protease comprises pepsin.

According to embodiments of the invention, the solubilizing is effected at a pH between 1.5-2.5.

According to embodiments of the invention, the solubilizing is effected at a pH between 1.5-3.5.

According to embodiments of the invention, the solubilizing is effected for 24-72 hours.

According to embodiments of the invention, the solubilizing is effected at a temperature between 20-25° C.

According to embodiments of the invention, the method further comprises homogenizing the decellularized omentum prior to the solubilizing.

According to embodiments of the invention, the homogenizing is effected under conditions that generate particulate matter having a surface area of between 2-100 μm².

According to embodiments of the invention, the homogenizing is effected under conditions that generate particulate matter having a surface area of between 2-12 μm².

According to embodiments of the invention, the conditions comprise a pH between 4.5-6.

According to embodiments of the invention, the decellularizing the omentum is effected using the method described herein.

According to an aspect of the present invention, there is provided a composition of matter generated according to the method described herein.

According to an aspect of the present invention, there is provided a method of regenerating tissue in a subject in need thereof, comprising administering to the subject a therapeutically effective amount of the composition of matter described herein, thereby regenerating the tissue in a subject.

According to embodiments of the invention, the omentum is autologous to the subject.

According to embodiments of the invention, the omentum is non-autologous to the subject.

According to embodiments of the invention, the tissue is neuronal tissue.

According to an aspect of the present invention, there is provided a method of generating transplantable material comprising particles: generating a liquid composition according to the method described herein; contacting the liquid composition with a population of cells; dispensing droplets of the liquid composition and the population of cells onto a solid surface; and subjecting the droplets to conditions that promote solidification of the droplets.

According to embodiments of the invention, the population of cells comprises pluripotent stem cells.

According to embodiments of the invention, the pluripotent stem cells comprise induced pluripotent stem cells.

According to embodiments of the invention, the induced pluripotent stem cells are reprogrammed from peripheral blood mononuclear cells (PBMC).

According to embodiments of the invention, the method further comprises differentiating the population of cells into mature neuronal cells and/or neural progenitor cells.

According to embodiments of the invention, the particles have a diameter between 750 microns-3 mm.

According to embodiments of the invention, the particles have a diameter between 400 microns-3 mm.

According to an aspect of the present invention, there is provided a transplant generated according to the method described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B. Macroscopic images of human and porcine omentum prior to decellularization. Fresh porcine omentum (1A); Fresh human omentum (1B).

FIG. 2 is a graph comparing DNA removal from decellularized human tissue by 1M NaCl vs. Benzonase endonuclease. Results are presented as mean ng DNA per mg dry tissue (n=7).

FIG. 3 is a flow-chart describing main steps of the decellularization of human omentum.

FIGS. 4A-I. Composition and matrix morphology of decellularized Omentum. DNA staining with Hoechst 33258 (blue) of native human omentum (A) and decellularized human ECM (B). Lipids staining with Oil Red O of native human omentum (C), and decellularized human ECM (D). Glycosaminoglycans (GAGs) staining with Alcian blue (light blue) of native human omentum (E), and decellularized human ECM (F). Scale bar =100 μm. Comparison of decellularized human and porcine ECM proteins composition (G) (n>4). Representative SEM images of the ECM obtained following decellularization of porcine (H) and human omentum (I).

FIGS. 5A-B. Macroscopic images of human and porcine omentum following decellularization. Lyophilized decellularized porcine omentum (A); Lyophilized decellularized human omentum (B).

FIGS. 6A-H. Gelation of human and porcine hydrogel. Macroscopic images of human and porcine hydrogel in liquid and solid form (A). Turbidity gelation kinetics of human- and porcine-based hydrogels. Normalized absorbance/Optical Density (OD) (B, grey human, black porcine) and time at 50% of maximum absorbance T50 (C), n≥2. Rheological properties of the human-and porcine-based hydrogels. Representative curves of complex viscosity vs. time during gelation at 37° C. (D); Representative curves of loss and storage modulus vs. time during gelation at 37° C. (E); Maximal complex viscosity (F); Maximal storage modulus (G); gelation time (H) n≥2.

FIGS. 7A-C. Omentum based-hydrogel microstructure. Representative SEM images of human and porcine hydrogels after gelation (A, B). Analysis of fiber diameter (nm) of human and porcine hydrogels (C).

FIGS. 8A-I. Omentum based-hydrogel biochemical content. Representative immunofluorescent images of human and porcine hydrogels stained for Collagen I (A, B), Collagen IV (C, D), Fibronectin (E, F) and Laminin (G, H). Comparison of sGAGs concentration in human-and porcine-based hydrogels (I). Results are presented as mean μg sulfated GAGs per mL hydrogel, n≥2.

FIGS. 9A-E. Omentum based-hydrogel cytocompatibility. Representative images of 3T3 cells attachment to human and porcine hydrogels 1 h post seeding (A, B) and migration depth of the cells 24 h post seeding (C, D). Comparison of metabolic activity and proliferation using Presto Blue of hNDF cells seeded on human and porcine hydrogels (E), n=2.

FIG. 10 is flow cytometry results of the iPSCs used in the transplants showing expression of OCT3/4, SSEA4, SSEA5 and SSEAl at day 0 and 24 of the differentiation process.

FIGS. 11A-H are photographs of immunofluorescently stained iPSCs expressing pluripotent marker OCT3/4 (green) at days 0 (A & B), day 4 (C), day 13 (D), day 19 (E) and day 25 (F) of the differentiation process. Proliferation of iPSCs represented by Ki67 at day 0 (G) and day 25 (H) of the differentiation process. Nuclei stained with Hoechst (blue).

FIGS. 12A-F are photographs of immunofluorescently stained differentiated transplants. A, B and C, Cells express neuronal markers (TUJI; green), and D (NeuN; green), Nuclei stained with Hoechst (blue). E, Cells express late motoneuron specific marker (HB9; pink, TUJ1; green, Nuclei; blue). F, Cells express the vesicular acetylcholine transporter located in pre-synaptic motoneuron vesicles (VACHT; pink, TUJ1; green, Nuclei; blue).

FIG. 13 is a heatmap of gene expression at days 14 and 21 during differentiation. Red color represents upregulation and blue color represents downregulation.

FIGS. 14A-C are photographs illustrating transplant potential functionality. A, Neurite outgrowth from a differentiated particle, cultured on a Geltrex-coated surface. B, Neurite-branched network formed between neighboring particles. C, TUJ1 staining of branched neurites.

FIG. 15 is a photograph of a droplet of hydrogel, generated according to embodiments of the invention.

FIGS. 16A-D illustrate turbidity gelation kinetics of human-and porcine-based hydrogels. Absorbance (A, grey porcine, black human), time at 50% of maximum absorbance T50 (B), Span of OD change (C) and calculated area under curve (AUC) (D). n=2.

FIG. 17 Proteomic LC-MS/MS analysis of human- and porcine-based hydrogels showing comparison of their protein composition.

### DESCRIPTION OF SPECIFIC EMBODIMENTS OF THE INVENTION

The present invention, in some embodiments thereof, relates to an improved method for decellularizing omentum, decellularized omentum matrices for tissue engineering and gels comprising decellularized omentum.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Omentum-based matrices fabricated by decellularization have the potential to serve as autologous scaffolds for tissue engineering.

The present inventors have now demonstrated an improved method for decellularizing omentum that can be used for generating cell scaffolds and as the source material for cell-supporting hydrogels. The present inventors developed a designated process, which was designed to eliminate all agents that present either functionality or safety risks, such as DNA and fat, while preserving structural and functional motifs of the ECM.

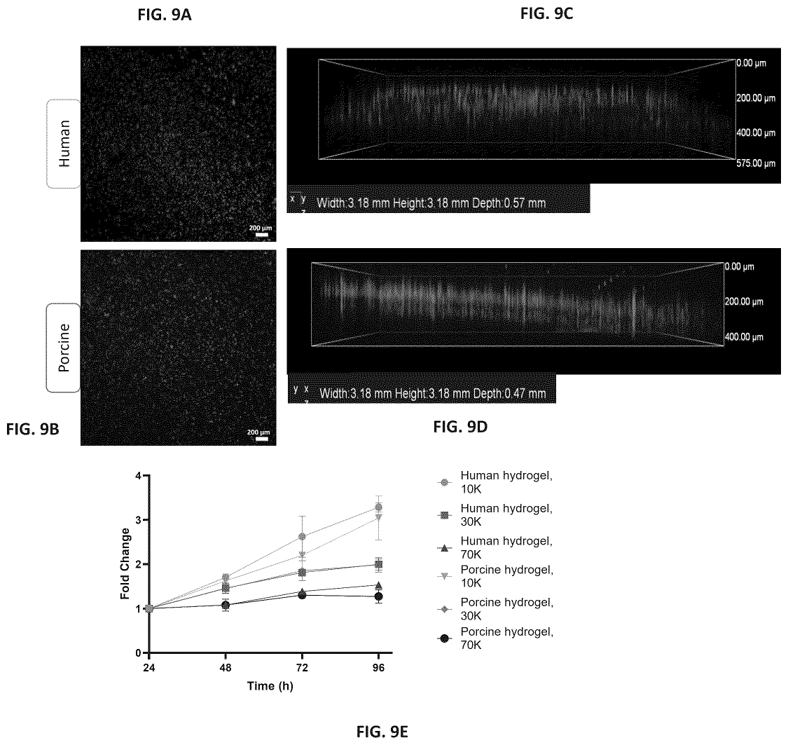

Whilst reducing the present invention to practice, the present inventors demonstrated that fabricated human omentum based-hydrogel (decellularized according to the methods described herein) could support cell attachment, migration and proliferation (FIGS. 9A-E), and therefore has the potential to serve as the scaffolding material for cells and tissues, offering an alternative to animal-sourced materials. The fact that the ECM originates from a fatty tissue that could be relatively easily and safely harvested presents the potential of using not only human-derived tissue as a source of biomaterial but rather the ability to use one's own body as the source for engineering a tissue.

The present inventors further demonstrated that omentum decellularized according to the improved method can be used to fabricate particles in which pluripotent stem cells can be differentiated. Depending on the lineage to which the cells are differentiated, the particles may be used to treat a myriad of disorders associated with tissue degeneration.

Thus, according to one aspect of the present invention, there is provided a method of decellularizing omentum comprising:
a. cutting the omentum into pieces having a surface area between 25-80 mm² and a volume of 13-70 mm³;
b. exposing the omentum to a hypotonic solution following step (a);
c. dehydrating the omentum following step (b);
d. extracting fat from the dehydrated omentum using polar and non-polar solvents following step (c);
e. rehydrating the dehydrated omentum following step (d);
f. removing cell debris from the rehydrated omentum using an enzyme selected from the group consisting of TrypLE™ Select, TrypLE™ Express and Trypsin-EDTA following step (e); and
g. degrading nucleic acid from the rehydrated omentum following step (f) using the endonuclease Benzonase® or Denarase®, thereby generating decellularized omentum.

According to another aspect of the present invention there is provided a method of decellularizing omentum comprising:
cutting the omentum into pieces having a surface area between 25-150 mm² and a volume of 13-125 mm³; exposing the omentum to a hypotonic solution following step (a);
dehydrating the omentum following step (b);
extracting fat from the dehydrated omentum using polar and non-polar solvents following step (c);
(e) rehydrating the dehydrated omentum following step (d);
(f) removing cell debris from the rehydrated omentum using an enzyme selected from the group consisting of TrypLE™ Select, TrypLE™ Express and Trypsin-EDTA following step (e); and
(g) degrading nucleic acid from the rehydrated omentum following step (f) using endonuclease Denarase®, thereby generating decellularized omentum.

Omentum may be harvested from mammalian species, such as humans, swine, bovine, caprine and the like.

According to a preferred embodiment, the omentum is derived from a human.

Following tissue harvesting, the tissue can be placed in an appropriate buffer (e.g., saline or PBS) for immediate processing or stored for later use, preferably at a temperature of about −20° C. to about −80° C.

The tissue is then cut (or chopped) using a blade (e.g., a scalpel or surgical scissors) into pieces. Preferably, the tissue is handled in such a way that preserves the overall structure of the ECM (e.g., the tissue is not sheared or crushed). The size of the tissue pieces, (also referred to herein as tissue portions or fragments) is such that they have a surface area between 25-150 mm² and a volume of 13-125 mm³. The size of the fragments may be approximated by eye (or any other measuring apparatus, such as a ruler) and the volume or surface area may be calculated according to whether the fragment more closely resembles a sphere or a cube. For example, if the fragment most closely resembles a sphere, and the diameter is typically between 2-5 mm, a volume and a surface area may be calculated. If the fragment most closely resembles a cube, and the side of one cube is between 2-5 mm, a volume and a surface area may be calculated. Preferably, at least 50%, 60%, 70%, 80%, 90% or even 95% of the fragments have a diameter/side within the 2-5 mm range.

The pieces are then placed in a hypotonic solution. A hypotonic solution is one in which the concentration of electrolytes is below that in cells. In this situation, osmotic pressure leads to the migration of water into the cells, in an attempt to equalize the electrolyte concentration inside and outside the cell walls.

Preferably, the hypotonic buffer used by the method according to this aspect of the present invention is 10 mM Tris solution at a pH of about 8.0 and includes approximately 0.1% (w/v) EDTA (5 mM EDTA).

The hypotonic buffer may comprise additional agents such as serine protease inhibitors (e.g. phenylmethanesulfonylfluoride or phenylmethylsulfonyl fluoride, PMSF) and/or anionic detergents such as sodium dodecyl sulphate (SDS).

According to this aspect of the present invention, the tissue is subjected to the hypotonic buffer for a time period leading to cell cytolysis, i.e., cell swelling and rupture (e.g., about an hour).

Following hypotonic shock, the tissue may optionally be subjected to cycles of freeze-thawing.

The freeze/thaw process preferably comprises freezing the tissue at, for example between −10 to −80° C., and typically at −80° C. for between 0.5-24 hours or between 2-24 hours and subsequently defrosting the tissue for about 0.5, 1, 2, 3 or 4 hours until it reaches room temperature or above (for example at 37° C.). This process is carried out at least once and preferably twice or three times in the presence of a hypotonic buffer.

Dehydration involves treating the omentum with one or more dehydration solvents, such one or more treatments of the omentum with a dehydration solvent(s) and/or such solvent(s) in solution with water. The one or more treatments may be sequential steps in the method performed with solutions having different ratios of dehydration solvent(s) to water, such as having gradually reduced amounts of water in the solution for each successive treatment and the final treatment may involve the use of pure solvent, i.e., solvent not in solution with water.

Low molecular weight organic solvents may be used for the dehydration solvent. In an embodiment, the dehydration solvent is one or more alcohols, such as those selected from the group consisting of methanol, ethanol, isopropanol, propanol and combinations thereof.

According to a particular embodiment, the omentum is dehydrated by rinsing once with 70% ethanol (for example for 10-60 minutes, about 15 minutes) and two to three times in 100% ethanol for 10-60 minutes each (e.g., about 15 minutes).

After dehydration, the fat may be extracted from the omentum using at least one polar solvent and one non-polar solvent, which may occur in one or more extraction steps so as to produce a composition that is devoid of lipids.

Examples of non-polar solvents are non-polar organic solvents such as hexane, xylene, benzene, toluene, ethyl acetate and combinations thereof. Polar solvents useful for the extraction solvent include acetone, dioxane, acetonitrile and combinations thereof. In an embodiment, the extraction solvent is selected from acetone, hexane, xylene and combinations thereof. Nonpolar solvents, include for example hexane, xylene and combinations thereof.

Fat extraction may be conducted in fat extraction steps by contacting the dehydrated omentum with the extraction solvents for varying periods of time.

Preferably, the polar lipids of the tissue are extracted by washing in the polar extraction agent (e.g., 100% acetone) between 10 minutes to 60 minutes. This may be repeated a number of times, or may be performed a single time. Then, the nonpolar lipids may be extracted by incubating in a mixture of nonpolar: polar agents (e.g., 60/40 (v/v) hexane: acetone solution (e.g. with 4 changes, three incubations of 1.5 hours and one incubation of 16 hours).

The phrase “devoid of lipids” as used herein refers to a composition comprising less than 10%, 9%, 8%, 7%, 6%, 5%, 4%, 3%, 2%, 1% of the lipids present in the natural (e.g., native) omentum.

After the fat extraction, the defatted, omentum is optionally re-hydrated. The defatted omentum may be re-hydrated by contacting the defatted, omentum with a re-hydration solvent, such as alcohol or a solution of alcohol in water, such as an alcohol solution having from about 60% to about 70% alcohol. Low molecular weight alcohols, such as methanol, ethanol, isopropanol, propanol and combinations thereof may be used. In an exemplary embodiment, the defatted, human omentum is washed once with 100% ethanol followed by three incubations with 70% ethanol.

The defatted, omentum is then decellularized by enzymatic proteolytic digestion which digests cellular components within the tissue yet preserves the ECM components (e.g., collagen and elastin) and thus results in a matrix which exhibits the mechanical and structural properties of the original tissue ECM.

Enzymatic digestion is preferably carried out using one of the following enzymes-recombinant trypsin, TrypLE™ Select or TrypLE™ Express.

Preferably, while in the digestion solution, the tissue segments are agitated (e.g., at about 150 rpm) to enable complete penetration of the digestion solution to the tissue. Preferably, the tissue segments are digested for at least 1 hour e.g., 1.5 hours.

The method according to this aspect of the present invention optionally and preferably includes a washing step and a subsequent additional step of removing nucleic acids (as well as residual nucleic acids) from the tissue to thereby obtain a nucleic acid-free tissue.

As used herein, the phrase “nucleic acid-free tissue” refers to a tissue being more than 99% free of any nucleic acid or fragments thereof as determined using conventional methods (e.g., spectrophotometry, electrophoresis). Such a step utilizes an endonuclease enzyme such as Benzonase® or Denarase®.

The above described endonuclease-comprising solution is preferably removed by subjecting the matrix to several washes in water or saline (e.g., at least 3 washes), until there is no evidence of detectable endonuclease in the matrix. Exemplary tests to ensure there is no residual Benzonase® or Denarase® are known in the art including for example ELISA (see Example 3, herein below).

Optionally, the decellularized omental ECM is then sterilized. Sterilization of the decellularized omental ECM may be affected using methods known in the art. In an embodiment, the decellularized omentum is contacted with a disinfection solution for a sufficiently effective period of time to disinfect the decellularized omentum, such as at least about 0.5 hour, typically about 1 hour to about 12 hours. The decellularized omentum may be fully submerged in the disinfection solution. The disinfection solution may comprise alcohol, or an alcohol in water solution, and may also include acid. The disinfection solution may include one or more of the following ethanol, methanol, isopropanol, propanol, hydrogen peroxide, peracetic acid and combinations thereof. In an embodiment, the disinfection solution has ethanol, such as 70% ethanol solution. Optionally, the decellularized omentum can be washed one or more times with ultrapure water.

It will be appreciated that if disinfection is not carried out, the process of decellularization may be carried out under aseptic conditions in order to maintain sterility.

Following the washing steps (and the optional sterilization process), the decellularized omentum may then be frozen, for example at temperatures between −20° C. or −80° C., preferably −20° C.

The present inventors have shown that decellularizing omentum according to the methods described herein results in the generation of decellularized omentum wherein more than 50% (or even more than 60%, 65%, 70%, 75%, 80%, 85%, 90%) of the total protein content thereof is collagen (i.e., total collagen-including collagen type I, II, III, IV, V, VI). In a particular embodiment, more than 40% of the total protein content is collagen type I.

As used herein the phrase “decellularized omentum” refers to the extracellular matrix which supports omentum tissue organization which has undergone a decellularization process (i.e., a removal of all cells from the tissue) and is thus devoid of cellular components.

The decellularized omentum obtained according to the presently described methods comprises less than 20% of the cells as compared to the number of cells (per volume or per weight) in the omentum prior to decellularization, more preferably less than 15% of the cells as compared to the number of cells (per volume or per weight) in the omentum prior to decellularization, more preferably less than 10% of the cells (per volume or per weight) as compared to the number of cells in the omentum prior to decellularization, more preferably less than 5% of the cells (per volume or per weight) as compared to the number of cells in the omentum prior to decellularization, more preferably less than 2% of the cells (per volume or per weight) as compared to the number of cells in the omentum prior to decellularization.

The phrase “devoid of cellular components” as used herein refers to being more than 90%, 91%, 92%, 93%, 94%, 95%, 96%, 97%, 98%, 99%, (e.g., 100%) devoid of the cellular components present in the natural (e.g., native) omentum. As used herein, the phrase “cellular components” refers to cell membrane components or intracellular components which make up the cell. Examples of cell components include cell structures (e.g., organelles) or molecules comprised in same. Examples of such include, but are not limited to, cell nuclei, nucleic acids, residual nucleic acids (e.g., fragmented nucleic acid sequences), cell membranes and/or residual cell membranes (e.g., fragmented membranes) which are present in cells of the tissue. It will be appreciated that due to the removal of all cellular components from the tissue, such a decellularized matrix cannot induce a cell-based immunological response when implanted in a subject.

The phrase “extracellular matrix (ECM)” as used herein, refers to a complex network of materials produced and secreted by the cells of the tissue into the surrounding extracellular space and/or medium and which typically together with the cells of the tissue impart the tissue its mechanical and structural properties. Generally, the ECM includes fibrous elements (particularly collagen, elastin, or reticulin), cell adhesion polypeptides (e.g., fibronectin, laminin and adhesive glycoproteins), and space-filling molecules [usually glycosaminoglycans (GAG), proteoglycans].

Typically, the decellularized omentum according to this aspect of the present invention comprises less than 50 ng DNA per mg dry decellularized omentum, more preferably less than 45 ng DNA and even more preferably less than 40 ng DNA per mg dry decellularized omentum.

As mentioned, the mean fiber diameter of the decellularized omentum is typically between about 70 nm-2 μm. In another embodiment, the mean fiber diameter of the decellularized, omentum is typically between about 1 μm-2 μm.

As used herein the term “porosity” refers to the three-dimensional measurement of empty space or void volume per total volume.

Typically, the open space of the composition following decellularization is greater than 50%, more preferably greater than 60%, more preferably greater than 70% and even more preferably greater than 95% as measured by SEM.

The phrase “devoid of lipids” as used herein, refers to a composition comprising less than 10%, 9%, 8%, 7%, 6%, 5%, 4%, 3%, 2%, 1% of the lipids present in the natural (e.g., native) omentum.

As mentioned, the decellularized omentum of this aspect of the present invention may be applied in tissue engineering and regeneration of internal organs, such as heart, kidney, liver, spleen and bladder. The decellularized omentum can also be used for repair and regeneration of skeletal tissues, such as bone, cartilage and tendon. Other uses for the decellularized omentum include soft tissue reinforcement and repair in combination with biocompatible meshes, such as dural grafting, hernia repair, and pelvic floor repair; nerve regeneration, such as a tubular structure for peripheral nerve regeneration; tissue augmentation; delivery of cells and bioactives; chronic wound repair; and bone repair. These uses and applications of the decellularized omentum are illustrative of several potential uses and should not be construed as limiting the types of uses and applications for the decellularized omentum prepared by the methods and processes described herein.

The decellularized omentum can be combined with synthetic constructs to make reinforced constructs. For example, the decellularized omentum matrix can be used as a scaffold structure for implantation in a mammalian body, such as scaffold for tissue repair. It can be further enhanced by bioactives, cells, small molecules, minced tissue and cell lysates.

As used herein, the term “scaffold” refers to a 3-dimensional matrix upon which cells may be cultured (i.e., survive and preferably proliferate for a predetermined time period).

The scaffold of this aspect of the present invention may be composed solely of decellularized omentum or may comprise additional polymers.

Thus, in other embodiments, the structural scaffold materials further comprise a “bioerodible” or “biodegradable” polymer or material.

The phrase “biodegradable polymer” as used herein, refers to a polymer or polymers which degrade in vivo, and wherein erosion of the polymer or polymers over time occurs concurrent with or subsequent to release of the components of the decellularized ECM. The terms “biodegradable” and “bioerodible” are equivalent and are used interchangeably herein.

Such bioerodible or biodegradable scaffold materials may be used to fabricate temporary structures. In exemplary embodiments, biodegradable or bioerodible structural scaffold materials may be biocompatible. Examples of biocompatible biodegradable polymers which are useful as scaffold materials include, but are not limited to, polylactic acid, polyglycolic acid, polycaprolactone, and copolymers thereof, polyesters such as polyglycolides, polyanhydrides, polyacrylates, polyalkyl cyanoacrylates such as n-butyl cyanoacrylate and isopropyl cyanoacrylate, polyacrylamides, polyorthoesters, polyphosphazenes, polypeptides, polyurethanes, polystyrenes, polystyrene sulfonic acid, polystyrene carboxylic acid, polyalkylene oxides, alginates, agaroses, dextrins, dextrans, polyanhydrides, biopolymers such as collagens and elastin, alginates, chitosans, glycosaminoglycans, and mixtures of such polymers. In still other embodiments, a mixture of non-biodegradable and bioerodible and/or biodegradable scaffold materials may be used to form a biomimetic structure of which part is permanent and part is temporary.

Therapeutic compounds or agents that modify cellular activity can also be incorporated (e.g., attached to, coated on, embedded or impregnated) into the scaffold material.

Campbell et al (US Patent Application No. 20030125410) which is incorporated by reference as if fully set forth by reference herein, discloses methods for fabrication of 3D scaffolds for stem cell growth, the scaffolds having preformed gradients of therapeutic compounds. The scaffold materials, according to Campbell et al, fall within the category of “bio-inks”. Such “bio-inks” are suitable for use with the compositions and methods of the present invention.

Exemplary agents that may be incorporated into the scaffold of the present invention include, but are not limited to those that promote cell adhesion (e.g., fibronectin, integrins), cell colonization, cell proliferation, cell differentiation, cell extravasation and/or cell migration. Thus, for example, the agent may be an amino acid, a small molecule chemical, a peptide, a polypeptide, a protein, a DNA, a RNA, a lipid and/or a proteoglycan.

Proteins that may be incorporated into the scaffolds of the present invention include, but are not limited to extracellular matrix proteins, cell adhesion proteins, growth factors, cytokines, hormones, proteases and protease substrates. Thus, exemplary proteins include vascular endothelial-derived growth factor (VEGF), activin-A, retinoic acid, epidermal growth factor, bone morphogenetic protein, TGFβ, hepatocyte growth factor, platelet-derived growth factor, TGFα. IGF-I and II, hematopoetic growth factors, heparin binding growth factor, peptide growth factors, erythropoietin, interleukins, tumor necrosis factors, interferons, colony stimulating factors, basic and acidic fibroblast growth factors, nerve growth factor (NGF) or muscle morphogenic factor (MMP). The particular growth factor employed should be appropriate to the desired cell activity. The regulatory effects of a large family of growth factors are well known to those skilled in the art.

The present invention contemplates seeding any cell type on the scaffolds described herein.

The cells may be derived from any organism including for example mammalian cells, (e.g., human), plant cells, algae cells, fungal cells (e.g., yeast cells), prokaryotic cells (e.g., bacterial cells).

According to a particular embodiment the cells comprise stem cells—e.g., adult stem cells such as mesenchymal stem cells or pluripotent stem cells such as embryonic stem cells or induced pluripotent stem cells (iPSCs). The stem cells may be modified so as to undergo ex vivo differentiation prior to seeding on the scaffold or may be seeded as pluripotent stem cells and further differentiated in situ prior to transplantation.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation, and cells originating from an unfertilized ova which are stimulated by parthenogenesis (parthenotes).

It will be appreciated that commercially available stem cells can also be used according to some embodiments of the invention. Human ES cells can be purchased from the NIH human embryonic stem cells registry [www(dot)grant (dot) nih(dot) gov/stem_cells/registry/current(dot) htm]. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUESI, NYUES2, NYUES3, NYUES4, NYUES5, NYUES6, NYUES7, UCLA 1, UCLA 2, UCLA 3, WA077 (H7), WA09 (H9), WA13 (H13), WA14 (H14), HUES 62, HUES 63, HUES 64, CT1, CT2, CT3, CT4, MA135, Encavour-2, WIBR1, WIBR2, WIBR3, WIBR4, WIBR5, WIBR6, HUES 45, Shef 3, Shef 6, BJNhem 19, BJNhem20, SA001, SA001.

Induced pluripotent stem cells (iPSCs; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as omentum) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kf14 and c-Myc/1-Myc in omental cells.

According to a particular embodiment, the cells are preferably intact (i.e., whole), and preferably viable, although it will be appreciated that pre-treatment of cells, such as generation of cell extracts or non-intact cells are also contemplated by the present invention.

The cells may be fresh, frozen or preserved in any other way known in the art (e.g., cryopreserved).

Cells can be seeded in a scaffold by static loading, or by seeding in stirred flask bioreactors (scaffold is typically suspended from a solid support), in a rotating wall vessel, or using direct perfusion of the cells in medium in a bioreactor. Highest cell density throughout the scaffold is achieved by the latter (direct perfusion) technique.

The cells may be seeded directly onto the scaffold, or alternatively, the cells may be mixed with a gel which is then absorbed onto the interior and exterior surfaces of the scaffold and which may fill some of the pores of the scaffold. Capillary forces will retain the gel on the scaffold before hardening, or the gel may be allowed to harden on the scaffold to become more self-supporting. Alternatively, the cells may be combined with a cell support substrate in the form of a gel optionally including extracellular matrix components. An exemplary gel is Matrigel™, from Becton-Dickinson. Matrigel™ is a solubilized basement membrane matrix extracted from the EHS mouse tumor (Kleinman, H. K., et al., Biochem. 25:312, 1986). The primary components of the matrix are laminin, collagen I, entactin, and heparan sulfate proteoglycan (perlecan) (Vukicevic, S., et al., Exp. Cell Res. 202:1, 1992). Matrigel™ also contains growth factors, matrix metalloproteinases (MMPs [collagenases]), and other proteinases (plasminogen activators [PAs]) (Mackay, A. R., et al., BioTechniques 15:1048, 1993). The matrix also includes several undefined compounds (Kleinman, H. K., et al., Biochem. 25:312, 1986; McGuire, P. G. and Seeds, N. W., J. Cell. Biochem. 40:215, 1989), but it does not contain any detectable levels of tissue inhibitors of metalloproteinases (TIMPs) (Mackay, A. R., et al., BioTechniques 15:1048, 1993). Alternatively, the gel may be growth-factor reduced Matrigel, produced by removing most of the growth factors from the gel (see Taub, et al., Proc. Natl. Acad. Sci. USA (1990); 87 (10:4002-6). In another embodiment, the gel may be a collagen I gel, alginate, or agar. Such a gel may also include other extracellular matrix components, such as glycosaminoglycans, fibrin, fibronectin, proteoglycans, and glycoproteins. The gel may also include basement membrane components such as collagen IV and laminin. Enzymes such as proteinases and collagenases may be added to the gel, as may cell response modifiers such as growth factors and chemotactic agents.

According to another embodiment, the decellularized omentum may be solubilized and formed into a hydrogel as further described herein below.

Thus, according to another aspect of the present invention there is provided a method of generating a liquid composition of matter suitable for tissue generation, which, upon temperature activation, is capable of solidifying, the method comprising:

decellularizing omentum to generate decellularized omentum; an

solubilizing the decellularized omentum whilst in a wet state, thereby generating the composition of matter.

The use of the hydrogel described herein has several advantages over solid decellularized omentum. For example, in a liquid form there is better control of the density and uniformity of the formed tissue since it is possible to control gel concentration, the uniformity of mixing hydrogel/cells and to control the desired shape and size of the cast mixture. The hydrogel described herein can also be 3D printed allowing for the generation of complex structures including defined areas and/or use of a plurality of cell types.

The first step in the generation of the liquid composition is decellularizing omentum. A particular method for decellularizing human omentum is described herein above.

According to this aspect of the present invention, the decellularized omentum is used in a wet state i.e., non-lyophilized. In an exemplary embodiment, the decellularized omentum has not been subjected to a detergent wash, following removal of nucleic acids.

The wet, decellularized omentum is subjected to a homogenization process so as to bring about a uniform end-product. The size of the particulate material in the product following the homogenization is typically between 2-575 μm² in surface area, (e.g. between 2-300 μm², between 2-100 μm², between 3-12 μm²). Preferably at least 50%, 60%, 70%, 80% 90% of the particulate material is in the range of 2-100 μm² as measured by image analysis (e.g., using FIJI (ImageJ)). Typically, as part of the homogenization process, grinding of the dECM is carried out by milling at cryogenic temperatures maintained by liquid Nitrogen (LN₂), at a neutral pH of between 6.5-7.5, (e.g., 7). Alternatively, homogenization can be performed by bead beating and/or shearing at a pH between 4.5-6 (e.g., 5.5) and at a temperature between 22-30° C.; preferably 25° C.

Once homogenized, the wet decellularized omentum is subjected to proteolytic digestion. The digestion is affected under conditions that allow the proteolytic enzyme to digest and solubilize the ECM. Thus, according to one embodiment, the digestion is carried out in the presence of an acid (e.g., 10-30 mM HCl) so as to obtain a pH of about 1.5-3.5, 1.5-2.5, preferably pH 2-3.

Proteolytic digestion according to this aspect of the present invention can be effected using a variety of proteolytic enzymes which cleave only the telopeptide of collagen. Non-limiting examples of suitable proteolytic enzymes include trypsin, pepsin, and pancreatin which are available from various sources such as from Sigma (St Louis, MO, USA) and combinations thereof. Matrix-degrading enzymes such as matrix metalloproteinases are also contemplated.

According to a particular embodiment, the enzyme is pepsin (e.g., 1:10 relative to dry weight (±10%)).

In one embodiment, the concentration of pepsin used is about 10 mg/ml.

In one embodiment, the pepsin reaction is carried out at a temperature between 20-25° C.; preferably 22-24° C., e.g. about 23° C., for at least about 20 hours, more preferably, at least about 24 hours and more preferably about 48 hours.

In another embodiment, the pepsin reaction is carried out at a temperature of about 30° C. for a short period of time (e.g., between 20 minutes-1 hour).

Once the decellularized ECM is digested and solubilized, the pH of the solution is increased (e.g., using NaOH) so as to irreversibly inactivate the proteolytic enzyme (e.g., to about pH 7). The decellularized, solubilized omentum, at a final ECM concentration of between 0.3-2% w/v, preferably 0.5-1% w/v, may be stored at this stage at temperatures lower than 20° C.—for example 4° C. or −20° C. so that the decellularized ECM remains in solution.

The solubilized, decellularized omentum (generated as described herein above) typically has less than 100 μg sulfated Glycosaminoglycans (GAGs) per mL of hydrogel.

More than 40% of the total collagen content of the solubilized, decellularized human omentum is collagen type I, wherein less than 20%, less than 19%, less than 18%, less than 17%, less than 16%, or less than 15% of the total protein content, is the alpha I chain of type III collagen.

Typically, the solubilized, decellularized omentum is capable of forming a gel at a temperature above about 30° C., above about 31° C., above about 32° C., above about 33° C., above about 34° C., above about 35° C., above about 36° C., above about 37° C.

The hydrogel generated from the precursor composition described herein is viscoelastic, thermo-responsive, has low swelling ratio and is biocompatible and degradable.

The quality of the hydrogel may be verified prior to use. In one embodiment, the quality is verified (e.g. by microscopy) by ensuring that it produces droplets of comparable sizes (e.g. +/−10%) that have well-defined outlines and substantially similar texture (e.g. smooth).

Typically, the DNA (ng) content per dry weight of hydrogel is less than 50 ng per mg dry weight of hydrogel, less than 45 ng per mg dry weight of hydrogel, or even less than 40 or 30 ng per mg dry weight of hydrogel (e.g. between 15-25 ng/mg per dry weight of hydrogel). Typically, it comprises the following components: collagen type I, II, III, IV, V, VI, laminin, elastin, fibronectin and glycosaminoglycans (sulfated and non-sulfated).

According to a particular embodiment, the sulfated GAG content per mL of hydrogel is between 1-50 μg or more preferably between 1-40 μg, or even more preferably between 1-30 μg.

According to still another embodiment, the diameter of the fibers in the hydrogel is between 5-500 nm (for example between 20-400 nm).

The open space between fibers of the hydrogel is typically in the range of 70-95%.

The half time of gelation is typically between 8-20 minutes e.g. between 10-20 minutes.

The hydrogel composition may be administered into the body using an injecting device (e.g. needle, catheter) when it is in a liquid form. The hydrogel may contain and release growth factors or therapeutic agents (as described herein above) in a controlled manner and/or as a substrate/carrier for cells.

Thus, the present inventors consider administration of the liquid hydrogel either in the presence or absence of cell populations to patients. Such cell populations have been described herein above.

The hydrogel may undergo a process of 3D printing. The hydrogel (together with cells or in the absence of cells), in its liquid state may be printed by extrusion through an aperture (e.g., syringe) so as to form a thin line of biomaterial. The diameter of the aperture is typically between 0.1-0.7 mms. By varying the hydrogel's temperature, velocity of printing, surface temperature or concentration, various printed hydrogel diameters may be obtained, ranging from 100 μm to several millimeters.

Another contemplated use of the liquid hydrogel is as an encapsulating agent. Thus, the liquid hydrogel may be added to a polymerizing agent to generate a mixture for generating capsules.

The polymerizing agent of this aspect of the present invention is preferably water soluble and may include polymers such as chitosan and polymethacrylic acid or hydrogels composed of polysaccharides (such as alginate, hyaluronic acid and agarose) or other polymers such as poly ethylene glycol, (PEG), and poly hydroxyethyl methacrylate (HEMA).

According to a particular embodiment, the polymerizing agent is chitosan or alginate.

According to another embodiment, the polymerizing agent is alginate. Alginate is commercially available from a variety of sources—e.g., Novamatrix, Norway. The alginate may be of a viscosity less than 20 up until greater than 200 mPa·s with different G/M content (e.g., from less than 1 to greater than 1.5).

Typical ratios of volumes of polymerizing agent: decellularized ECM which are mixed to generate the mixture are between 50:50-70:30.

Cells are added to the above described mixture. Thus, for example for a 2 mL mixture, about two million cells may be added.

Decellularized omentum generated according to methods described herein may be used to generate particles. In this context, the term “particles” refers to a structure having a distinct shape (e.g. substantially round, or hemi-spherical) and having a size between 400 microns-3 mm. The particles are transplantable. A plurality of particles which are transplanted simultaneously to a particular site is referred to herein as a transplant.

Additional information on use of decellularized ECM as an encapsulating agent is provided in WO 2014/037942, incorporated herein by reference.

Another method of forming particles from solubilized, decellularized omentum relies on the formation of droplets as further described herein below.

Optionally, the solubilized, decellularized omentum is mixed with cells prior to formation of the droplets.

Exemplary cells that can be included in the particles are described herein above.

According to a particular embodiment, stem cells are used, including induced pluripotent stem cells or embryonic stem cells.

In one embodiment, the induced pluripotent stem cells are derived from peripheral blood mononuclear cells (PBMC).

In one embodiment, the induced pluripotent stem cells are derived from omental stromal cells.

According to a particular embodiment, the solubilized decellularized omentum is mixed with stem cells (e.g., dissociated colonies of iPSCs cells) at a volumetric ratio of 1:2 to 1:12 stem cell pellet: solubilized decellularized omentum.

Droplets of between 0.5-3 μl, e.g. between 1-3 μl, for example between 1.5-2.5 μl of the solution may be generated using an automated dispensing device onto a solid surface (e.g. silicon, glass or plastic). Other surface types are also envisaged including oil based surfaces and water based surfaces. The droplets are generated at a temperature which maintains the solubilized, decellularized omentum as a liquid. Once formed the droplets are then subjected to a temperature of above 30° C. (e.g., 37° C.) for at least 8, or at least 15 minutes to ensure that the droplets have solidified and form solid, gel-like particles. Upon gelation the particles are then cultured in a medium, such that the cells seeded therein remain viable.

It will be appreciated that since the cells are mixed with the decellularized omentum when it is in a liquid form (i.e., prior to particle formation) and not seeded upon the pre-formed particles, the cells are typically distributed homogeneously throughout the particles.

If pluripotent stem cells are included in the particles, the next step of the process includes differentiation towards the required cell type.

Prior to the differentiation step, the stem cells comprised in the particle may be allowed to proliferate to fill the volume-e.g., for at least 1 day, 2 days, 3 days, preferably 3 days. The particles are cultured in a medium which support the cells pluripotency. Typically, each particle comprises about 15,000-150,000 stem cells at the start of the differentiation step. In one embodiment, the differentiation step is started when the cells reach about 90% confluence.

The subsequent differentiation process may be carried out under gentle agitation to support mass/nutrient transfer and to ensure efficient penetration of the culture medium into the core of the particle.

In one embodiment, the particles are cultured in a medium comprising neuronal differentiating agents under conditions that promote diffusion of the neuronal differentiating agents into the particle.

Methods of differentiating pluripotent stem cells into neuronal cells are known in the art and include those disclosed by Edri et al., Advanced materials 31, 1803895 (2019); Shimojo. et al. Mol Brain 8, 79 (2015); Yiet al., Stem Cells International, 2018, Article ID 3628578; Faravelli et al. Stem Cell Research & Therapy, 2014, 5:87; Wada et al. PLOS One, 2009, Volume 4, Issue 8, c6722; Qu et al., Nature Communications,5:3449 |DOI: 10.1038/ncomms4449; Karumayaram et al., Stem Cells. 2009 April; 27(4): 806-811. doi: 10.1002/stem.31, the contents of each are incorporated herein by reference.

Methods of differentiating mesenchymal stem cells into cells of the neuronal lineage are provided for example in WO2006/134602, WO2009/144718, WO2007/066338 and WO2004/046348, the teachings of which are incorporated herein by reference.

Exemplary neuronal differentiation agents which can be used in the differentiation process include, but are not limited to retinoic acid, valproic acid and derivatives thereof (e.g., esters, salts, retinoids, retinates, valproates, etc.); thyroid hormone or other agonists of thyroid hormone receptor; noggin; BDNF, NT 4/5 or other agonists of the NTRK2 receptor; agents which increase expression of the transcription factors ASCL1, OLIG1; d113 agonists, Notch 1, 2, 3 or 4 antagonists, gamma secretase inhibitors, including small molecule inhibitors of nicastrin, Aph1A, Aph1B, Psen1, Psen2 and PSENEN, delta like ligand (D11)-1 antagonist, delta like ligand (D11)-Partiall4, jagged 1 antagonist, jagged 2 antagonist; numb agonist or numb-like agonist.

According to one embodiment, the culturing is carried out under conditions that promote differentiation of at least a portion (e.g. at least 60%, at least 70%, at least 80%, at least 90%, at least 95% or even 100%) of the cells in the particle to mature neurons (e.g., mature motor neurons) which form a neuronal network in the particle and preferably also between one particle and another. The particles may comprise a mixture of fully differentiated and neural progenitor cells.

In one embodiment, the differentiation process comprises
culture iPSCs in the presence of an ALK5 inhibitor, an ALK2/ALK3 inhibitor and a GSK3 inhibitor
subsequent culture in the presence of retinoic acid and a hedgehog pathway agonist (purmorphamine)
subsequent culture in the presence of sonic hedgehog and retinoic acid;
subsequent culture in the presence of a neurotrophic factor (e.g. BDNF), ascorbic acid, hedgehog pathway agonist (purmorphamine) and retinoic acid; an subsequent culture in the presence of a γ-secretase inhibitor (e.g., DAPT).

The particles may also comprise additional cells such as astrocytes.

The neurons may be excitatory neurons or inhibitory neurons.

In one embodiment, the neurons comprise motor neurons.

The neurons of this aspect of the present invention express markers indicative of mature neurons (e.g. express dendritic markers such as MAP2, markers for synapses (SYP) and markers for neuronal intermediate filaments (NFM)).

In another embodiment, the neurons express markers of mature motor neurons including, but not limited to choline acetyltransferase (ChAT), HB9 (also known as MNX1) and ISL-1.

The term “neuronal network” refers to a collection of interconnected neurons comprising dendrites and having synapses therebetween. In one embodiment, the neuronal network also comprises neurofilaments.

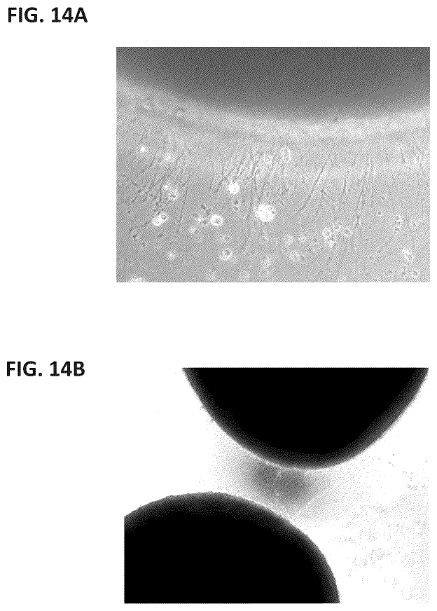

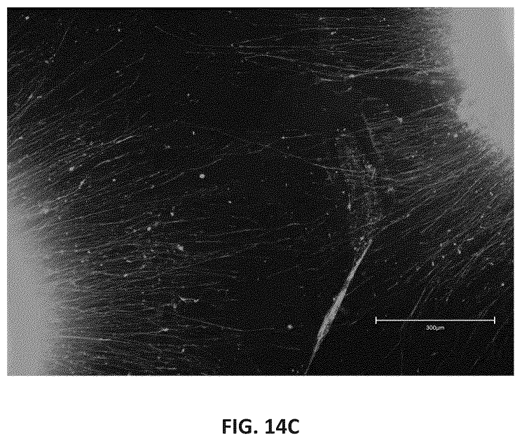

The neurons of the network in a particular particle may be capable of connecting with neurons of the network of another particle, under appropriate conditions. In one embodiment, the connections between the neurons of the two particles occurs following transplantation into the site of injury. In another embodiment, the connections between the neurons of the two particles can occur ex vivo (see for example FIG. 14B).

Methods of differentiating pluripotent stem cells into cell lineages other than neuronal cells are known in the art—see for example Abbar et al., BioResearch Open Access Volume 9.1, 2020; Lyra Leite et al., STAR Protoc. 2022 Aug. 18; 3(3):101560. doi: 10.1016/j.xpro.2022.101560. eCollection 2022 Sep. 16; Breunig et al, STAR Protoc. Volume 2, Issue 4, 17 Dec. 2021, 100913; Iberite et al., npj Regenerative Medicine volume 7, Article number: 23 (2022).

In any of the compositions described herein, the decellularized omentum may be derived from the patient him or herself (i.e., autologous to the patient) or derived from a subject other than the patient (i.e., non-autologous) and/or the cell populations which are administered to the patient together with the decellularized omentum are derived from the patient himself (i.e., autologous to the patient) or derived from a subject other than the patient (i.e., non-autologous).

The compositions of the present invention may be used for treating any disorder associated with tissue degeneration or damage. According to a specific embodiment, the compositions are used for treating a cardiac disorder which is associated with a defective or absent myocardium. According to another embodiment, the composition is used to treat nerve damage (due to injury or a disease, such as a neurodegenerative disease). In one embodiment, the composition of the present invention is used to treat a spinal cord injury. Furthermore, the composition may be used for generating organs and tissues by 3D printing (for drug screening and for therapy).

As used herein, the phrase “spinal cord injury” refers to an injury to the spinal cord that is caused by trauma instead of disease. Depending on where the spinal cord and nerve roots are damaged, the symptoms can vary widely, for example from pain to paralysis to incontinence. Spinal cord injuries are described at various levels of “incomplete”, which can vary from having no effect on the patient to a “complete” injury which means a total loss of function. Spinal cord injuries have many causes, but are typically associated with major trauma from motor vehicle accidents, falls, sports injuries, and violence. The abbreviation “SCI” means spinal cord injury.

The spinal cord injury may be susceptible to secondary tissue injury, including but not limited to: glial scarring, myelin inhibition, demyelination, cell death, lack of neurotrophic support, ischemia, free-radical formation, and excitotoxicity. This secondary tissue injury typically occurs at least 3 months, 4 months, 5 months, 6 months or later after the initial injury. This phase can also be referred to as chronic spinal cord injury.

The method according to this aspect of the present invention is affected by transplanting a therapeutically effective amount of the composition of the present invention to the subject (either together with the appropriate cells or without the cells). When the composition is in a liquid form (e.g., liquid hydrogel composition), it may be injected into the body at a preferable site. When the composition is in a solid form, it may be transplanted into the body at a preferable site.

As used herein, “transplanting” refers to providing the scaffold supported cells of the present invention, using any suitable route.

As used herein, a therapeutically effective dose is an amount sufficient to affect a beneficial or desired clinical result, which dose could be administered in one or more administrations. According to one embodiment, a single administration is employed.

It will be recognized by the skilled practitioner that when administering non-syngeneic cells or tissues to a subject, there is routinely immune rejection of such cells or tissues by the subject. Thus, the method of the present invention may also comprise treating the subject with an immunosuppressive regimen, preferably prior to such administration, so as to inhibit such rejection. Immunosuppressive protocols for inhibiting allogeneic graft rejection, for example via administration of cyclosporin A, immunosuppressive antibodies, and the like are widespread and standard practice in the clinic.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Materials and Methods

Decellularization of human omental tissue: Briefly, major blood vessels were manually removed from the tissue and the remaining tissue was chopped into 2-5 mm pieces with a scalpel. Samples were washed several times in PBS, before being agitated for 1 h in a hypotonic solution of 10 mM Tris 5 mM ethylenediaminetetraacetic acid (EDTA) at room temperature (RT). Then, in fresh hypotonic solution, the tissue was subjected to three cycles of freezing (−80° C.) and thawing (37° C.). After the last freeze-thaw cycle the tissue was gradually dehydrated by washing it once with 70% ethanol for 15 min and three times in 100% ethanol for 15 min each. Polar lipids of the tissue were then extracted by three 15 min washes of 100% acetone and a-polar lipids were extracted by four incubations in a 60:40 hexane: acetone solution (three incubations of 1.5 h and one incubation of 16 h). Then, the remaining tissue was gradually rehydrated and subjected to TrypLE (Thermo, cat #A1285901) degradation for 1.5 h at 37° C. The tissue was thoroughly washed with phosphate buffered saline (PBS) and with 50 mM Tris 1 mM MgCl₂. Afterwards, the tissue was gently agitated in a nucleic acid degradation solution of 50 mM Tris 1 mM MgCl₂ and 60 U/mL Benzonase® endonuclease EMPROVER EXPERT (Merck Millipore) for 20 h at 37° C. Finally, the tissue washed once with PBS and six times with sterile double distilled water (DDW). The decellularized tissue was stored frozen (−20° C.).

Preparation of solubilized human omentum dECM: The decellularized omentum was grinded and homogenized. Homogenized samples were enzymatically digested by adding a solution of pepsin (Merck≥2000 FIP units/mg protein) in 10 mM HCl (1 mg of pepsin enzyme per 10 mg of dry ECM) to reach a final dECM concentration of about 0.6% (w/v). The dECM was digested for 48 h at RT under constant stirring, until the liquid was homogenous with no visible particles. Subsequently, the salt concentration was adjusted using PBSx10 and the pH was raised to 6.8-7.4 using 1 M NaOH. Raising the pH terminates pepsin activity (the enzyme is deactivated above pH 6).

Gelation kinetics: Gelation kinetics was evaluated spectrophotometrically. Briefly, 100 μL of 4° C. hydrogels were transferred to 96-well plate in duplicates. Absorbance at 405 nm was measured every 30 s for 1 h using an Epoch 2 Microplate Spectrophotometer (BioTek), preheated to 37° C. Absorbance values were normalized and plotted over time. The half-time of gelation was defined as the time when the material reached 50% of the normalized maximum measured absorbance.

Sulfated glycosaminoglycan quantification: The sulfated glycosaminoglycans (GAGs) in the produced hydrogels were quantified using the Blyscan sulfated GAG assay kit (Biocolor Ltd, Carrickfergus, UK) according to the manufacturer instructions. Briefly, the hydrogels were digested with papain, centrifuged to remove undigested remains, and the supernatants were examined with dimethylmethylene blue in duplicates.

Rheological evaluation: Rheological measurements were performed as previously described by Nadav Noor (onlinelibrarydotwileydotcom/doi/pdf/10dot1002/advsdot201900344) using Discovery HR-3 hybrid Rheometer (TA Instruments, DE) with 8 mm diameter parallel plate geometry with a Peltier plate to maintain the sample temperature. The samples (100 μL) were loaded at a temperature of 4° C., which was then raised to 37° C. to induce gelation; during which the oscillatory moduli of samples were monitored at a fixed frequency of 0.8 rad s⁻¹ and a strain of 1%.

Proteomic LC-MS/MS analysis: Analysis was performed by Smoler Proteomics Center (Technion). Briefly, samples of human and porcine decellularized tissue (˜30 mg) were digested with trypsin, analyzed by LC-MS/MS on Q-Exactive HF (Thermo) and identified by Discoverer software against either the Sus scrofa or the human proteome from the Uniprot database, and a decoy database (in order to determine the false discovery rate). All the identified peptides were filtered with high confidence, and the identified proteins with a minimum of 2 peptides. Peptide percentage out of the sample was calculated by IBAQ.

Cytotoxicity/Proliferation Study: hNDF cells in complete growth medium (DMEM high glucose supplemented with 10% FBS, 1% P/S, 1% L-Glu, 1% NEAA and 0.2% 2-Mercaptoethano) were seeded in monolayer in 96-well plates, on plates coated with either human or porcine hydrogel. The hydrogel was solidified and crosslinked by 30 min incubation at 37° C. The cells were seeded in duplicates, in 3 concentrations: 10⁴, 3*10⁴ and 7*10⁴ cells per well. Following the seeding, the cells were incubated overnight to allow attach to the hydrogel.

Cell viability and proliferation were evaluated after 24, 48, 72 and 96 h, in duplicates. Briefly, for each time point the well was washed once with DMEM, then 0.2 mL of PrestoBlue™ Cell Viability Reagent (Invitrogen) diluted 1:10 in DMEM was added to each well, and plate was incubated in a humidified cell incubator with 5% CO₂ at 37° C. for 90 min. 0.18 mL of the supernatant was collected from each well to a new 96-well plate, and ΔOD at 570 nm-600 nm was measured (TECAN). Results are presented as ΔOD fold change from T=24 hr.

SEM of DC omentum (qualitative) and of hydrogel (fiber diameter, quantitative): Samples were fixed with 2.5% w/v glutaraldehyde in PBS (overnight (16-20 h, at 4° C.), followed by a graded incubation series in ethanol-water solutions (30-100% (v/v)). All samples were critical point dried. Samples were then mounted onto aluminum stubs with conductive paint and sputter-coated with an ultrathin (150 Å) layer of gold in a Polaron E 5100 coating apparatus (Quorum technologies, Laughton, UK). The samples were viewed under JCM-6000PLUS NeoScope Benchtop (JEOL USA Inc., Peabody, MA).

Staining of native and DC omentum: Oil red, Alcian Blue, Hoechst: For Oil red staining, samples were fixed in cold methanol (−20° C.) for 10 min, then washed three times with PBS and left to dry in a chemical hood. Samples were then stained with Oil red diluted solution (3:2 in DDW) for 10 min at RT, followed by extensive washes in PBS. Samples were visualized using an inverted microscope (Evos, Zeiss).

For GAG staining, the samples were fixed in cold acetone (−20° C.) for 10 min, then air dried in chemical hood. Samples were then hydrated by a graded incubation series in ethanol-water solutions (100-70% (v/v)) for 2 min each. Samples were incubated for 30 min in Alcian blue dye, following extensive washes. Samples were visualized using an inverted microscope (Nikon Eclipse Ti).

For nuclei detection, the samples were incubated for 3 min with Hoechst 33258 (5 μg/mL; Sigma) and washed three times with PBS. Samples were visualized under the same exposure time (200 ms) using an inverted fluorescence microscope (Nikon Eclipse Ti).

Staining of Hydrogel-Laminin, Fibronectin, and Collagen: Samples were fixed in 4% formaldehyde in PBS for 20 min, washed three times in PBS and then permeabilized by 1 h incubation at room temperature in PBS-based blocking buffer (containing 1% bovine serum albumin (BSA) and 10% fetal bovine serum (FBS)) with 0.05% triton x-100, after which the samples were washed three times. Next, samples were blocked for 10 min at room temperature in PBS-based blocking buffer and then washed three times. Samples were incubated with primary antibodies (Collagen I, MA1-26771, 1:4000; Collagen IV, ab6586, 1:500; Fibronectin, ab6328, 1:100; Laminin, ab11575, 1:500) diluted in blocking solution for 90 min, followed by incubation with appropriate secondary antibodies (Jackson, 111-545-144 and 115-605-003, 1:250) for additional 90 min at RT. The samples were imaged using an upright confocal microscope (Nikon ECLIPSE NI-E) and inverted fluorescence microscope (Nikon ECLIPSE TI-E). Images were processed and analyzed using the NIS elements software (Nikon Instruments). Representative images were chosen.

Cell attachment+Migration at 24 hr: For cell attachment and migration tests, 3T3 fibroblasts were pre-stained with Cytopainter cell proliferation staining reagent (Abcam; ab 176736) according to manufacturer's protocol. The fluorescent dye is absorbed by the cells and is transferred to daughter cells upon proliferation.

In parallel, 100 mL of omentum hydrogel (human or porcine, n=3 for each type) was evenly spread on the surface of 1.5 cm diameter culture plate. The hydrogel was incubated at 37° C. for 30 min. Then, 10⁵ pre-stained cells were seeded in each plate on the surface of the crosslinked hydrogel.

For cell attachment, the constructs were incubated for 1 h at 37° C., following 3 gentle washes. The cells were observed using an upright microscope (Nikon ECLIPSE NI-E).

For cell migration, the constructs were observed at the Y-Z axis using upright confocal microscope (Nikon). Images were processed and analyzed using the NIS elements software (Nikon Instruments) 24 h after cell seeding.

### Results

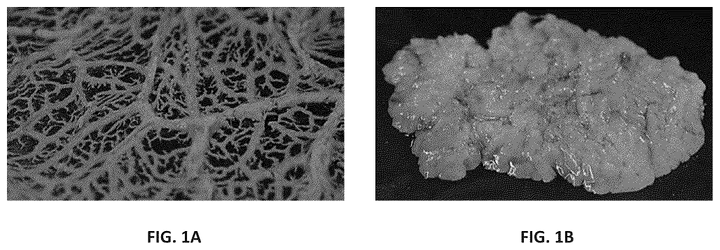

Decellularization of human omentum: As shown, the macroscopic morphology of porcine and human omentum is different (FIGS. 1A-B). While the porcine tissue (FIG. 1A) is transparent, with distinguishable blood vessels distributed throughout the tissue, the human omentum (FIG. 1B) is denser and orange, and blood vessel network is difficult to detect.

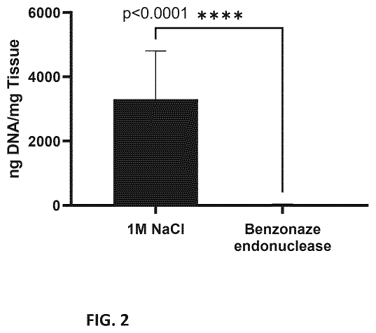

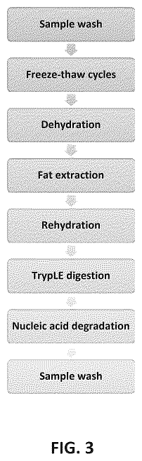

The porcine decellularization process was applied to the human omentum sample. Efficiency was evaluated in terms of DNA removal, which is considered a major parameter for process efficiency. As shown in FIG. 2, after applying the process on human omentum using 1M NaCl, DNA removal was inefficient, leaving higher DNA than the acceptable amount of 50 ng DNA per mg of dry tissue weight.

Various steps were taken in order to adapt the process for decellularization of human omentum.

The first step of the decellularization process is sample preparation. Since porcine omentum is very thin and spread, there is no actual need to mince the tissue. However, as the human sample is much thicker and denser, the first step of preparation was to increase the tissue surface area per volume. Therefore, the human sample was cut to ˜0.5×0.5cm pieces to ensure efficient exposure of the tissue to all reagents during the process.

At the next stage we used freeze-thaw cycles to promote cell lysis. Three cycles of freeze-thaw (−80° C.□37° C.), were performed, before starting the fat extraction stage.

Fat extraction was performed following an initial dehydration process and was followed by a rehydration process. Dehydration of human omentum, as well as rehydration cycles were done with significantly more fluid exchanges as compared to those used for porcine omentum to ensure optimal fat removal. The majority of lipids were extracted from the human omentum during the first hours.

The following step was designed to remove adhered cell debris. Using the porcine protocol (Trypsin-EDTA digestion for 16h at room temperature), the tissue texture was compromised and the appearance was transparent, and slippery which may indicate some degree of digestion. The protocol was therefore adapted, whereby the incubation was carried out at 37° C. for 1.5h, with TrypLE Select, which is a recombinant enzyme replacing animal trypsin, for the dissociation of adherent mammalian cells. The use of TrypLE Select resulted in ECM preservation without the risk of having traces of materials from an animal origin.

One of the crucial steps of decellularization is removal of residual DNA, which may provoke an immune response in the patient after transplantation. Such phenomenon could also have an effect when working with autologous hydrogels, in the form of autoimmune response. It is accepted that 50 ng DNA per mg of dry tissue weight should be the threshold. Preliminary attempts to apply the original porcine decellularization process on the human sample resulted in insufficient removal of DNA. The use of high salt concentration solution (1M NaCl) was not enough to extract sufficient amount of DNA from the human omentum sample, 3302±1506 ng DNA/mg dry sample of residual DNA was detected at the end of the process (FIG. 2). Attempts to increase the concentration of NaCl, incubation time and solution exchange, as well as to change the sequence of steps, did not lead to efficient removal of the DNA. Accordingly, a designated endonuclease digestion that degrades all forms of DNA and RNA (Benzonase, for 20 h at 37° C.) was tested and resulted in a very efficient removal of the DNA, yielding 28±17 ng DNA/mg dry sample of residual DNA (FIG. 2).

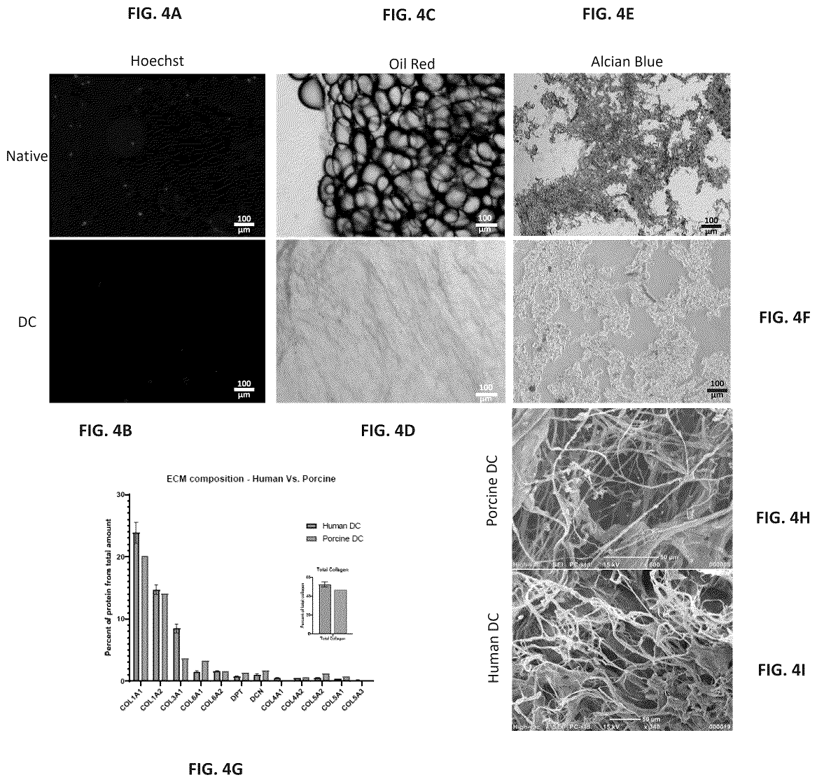

Characterization of the decellularized matrix: To qualitatively assess nucleic acid amounts in the decellularized human omentum, staining with Hoechst 33258, was performed. As shown, cells are clearly present in the sample prior to decellularization process. However, images taken with the same light exposure (duration and intensity) revealed efficient removal of the cells, as indicated by the lack of apparent staining (FIGS. 4A-B).

Prior to decellularization, a significant amount of fat, which appeared as red droplets by Oil Red staining, could be observed. Following decellularization process, red droplets could not be detected, indicating efficient fat extraction (FIG. 4C-D).

As presented in FIG. 4G, it is apparent that the vast majority of proteins within both human and porcine ECMs are collagens. Additional ECM proteins were detected. Interestingly, quantitative differences in collagen type I between human and porcine samples were detected. This can be attributed to intrinsic differences between human-and porcine-based samples. The expected differences in composition of human and porcine tissues emphasize the need of an adjusted decellularization process. Moreover, the porcine samples were obtained from relatively young individuals, while the human samples were obtained from older donors. It is well known that during tissue maturation a shift between collagen III, the more elastic collagen to the stiffer collagen I, occurs (Mays PK, 1988). The human ECM also contained many other ECM proteins that were not detected in the porcine samples. However, their intensity was significantly lower than that of collagens and was equivalent to less than 0.5% of the total proteins.

Glycosaminoglycan (GAG) content was evaluated prior to and following decellularization by Alcian Blue staining as shown in FIGS. 4E-F.

The structure of the human omentum-based ECM was evaluated by scanning electron microscopy (SEM). As shown in FIG. 4H, I, both porcine and human ECM have a fibrous structure, indicating that in spite of variations in the decellularization process, the human sample was efficiently processed and maintained a fibrous structure, suitable for ECM-cells interactions.

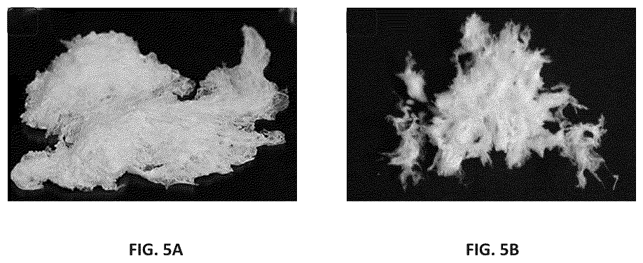

Fabrication of a human ECM-based hydrogel: The process of transforming the decellularized ECM into a thermo-responsive hydrogel includes dECM mincing or milling, followed by digestion with Pepsin in an acidic environment to solubilize the collagen by cleaving the telopeptides edges. Upon receiving a homogeneous solution, the Pepsin is inactivated. The original process of porcine hydrogel formation includes a lyophilization step. The lyophilization process of both porcine and human decellularized samples resulted in a dry white scaffolding material (FIGS. 5A-B). The human sample became very difficult to handle.

Many changes in the porcine protocol, including homogenization of a wet (non-lyophilized) dECM at a specific temperature or pH and under a controlled temperature with a defined ECM to fluid ratio and interval homogenization/dissociation, were implemented to accommodate the human sample. The latter was incubated with 1 mg of pepsin enzyme per 10 mg of dry ECM for 48 h at pH 2. Following completion of enzymatic digestion, the ECM was fully converted to a homogeneous solution. Following digestion, pepsin was inactivated and pH and osmolarity were adjusted to reach physiological conditions (pH 6.8-7.4; 275-295 mOsm per kilogram, respectively).

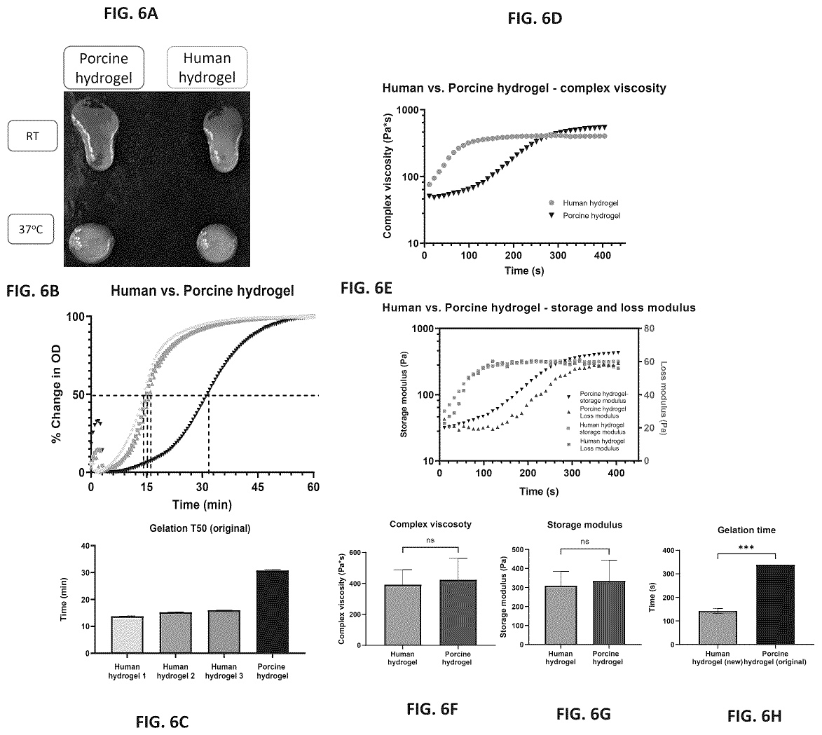

Characterization of the human ECM-based hydrogel: The formed hydrogel possesses the ability to remain a weak gel while at 4° C., and to solidify upon exposure to elevated temperature. The first step in the hydrogel assessment process was to visually examine gel formation after 30 min (FIG. 6A). As shown, both human and porcine hydrogels prior to solidification tend to leak on a tilted smooth surface, whereas following solidification, both preserve the original casted shape.

Gelation kinetics was evaluated using spectrophotometric turbidity assay (FIG. 6B-C). Changes in absorbance of liquid ECM during gelation period were used for calculating gelation kinetics parameters. Both human- and porcine-based hydrogels presented a typical elevation in absorbance (OD) as a function of time, upon exposure to 37° C. (sigmoidal graph). Gelation parameters calculated from the obtained graphs revealed different gelation kinetics. Faster gelation was obtained with human-based hydrogels compared to porcine-based hydrogels. This could be attributed to the difference in the intrinsic features of the original ECM, in terms of different protein composition.

When the temperature was elevated from 4° C. to 37° C. and during the gelation period, storage modulus (G′), loss modulus (G″) and complex viscosity changed over time, and were characterized by a sigmoidal shape (FIG. 6D-E). The storage modulus was greater than the loss modulus throughout the measurements, indicating elastic properties of the gel. Additionally, although the final storage modulus of human-based hydrogel was relatively similar to the porcine's (335±110 Pa vs. 309±76 Pa, respectively) as well as the complex viscosity (392±97 Pa*s vs. 423±139 Pa*s, respectively) (FIGS. 6F-G), the kinetics were different, porcine hydrogel rate was slightly slower compared to that of human hydrogel, which was in line with the gelation time (FIG. 6H) and kinetics, obtained by the turbidity assay (FIG. 6B-C). These results reflect the variation of the cross-linking process that is a direct result of the different composition of the hydrogels as well as the process.

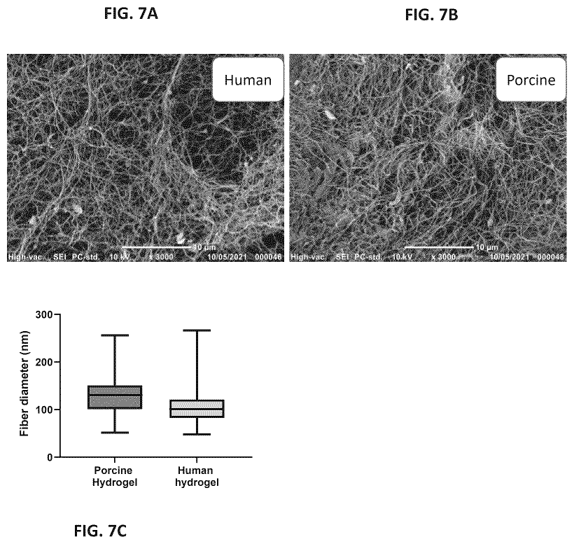

The structure of the fibers within the hydrogel was evaluated by SEM. As shown (FIG. 7A-B), both the human and porcine hydrogels had similar fibrous morphology with fiber diameter of 106±34 nm, and 128±37 nm, respectively.

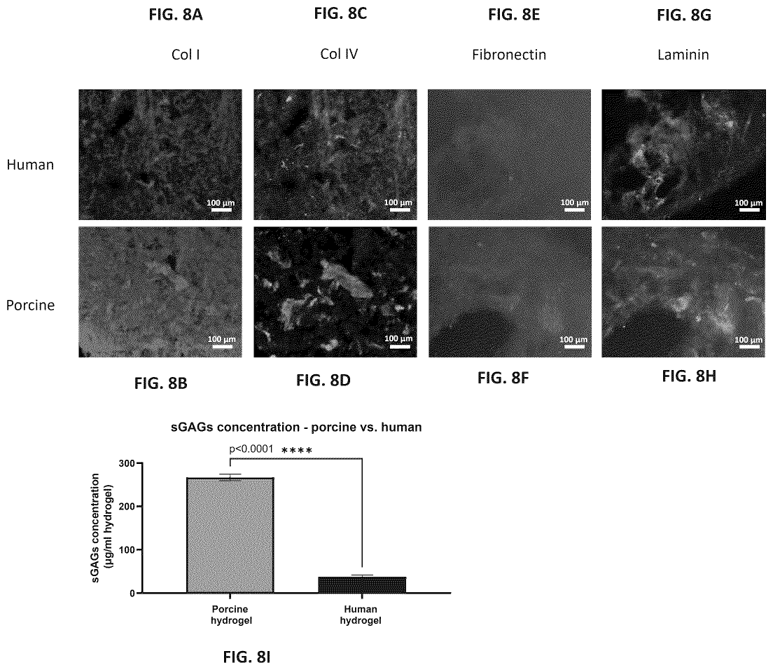

As shown, both porcine and human hydrogels present the main ECM proteins Collagen type I, Collagen Type IV, fibronectin and laminin which further support that the proposed decellularization as well as the hydrogel generation processes preserved the ECM proteins (FIGS. 8A-H).

Blyscan assay revealed lower levels of sulfated GAGs within the human hydrogel, as compared to the porcine sample (FIG. 8I), probably due to the differences in the decellularization process.

Fibroblast attachment to the human and porcine hydrogel was assessed one hour post seeding, revealing uniform cell attachment to both porcine and human hydrogel (FIG. 9A-B). Twenty-four hours post seeding, the migration of human fibroblasts into the human hydrogel was assessed. As shown, the cells were able to penetrate to a similar depth in the human hydrogel and the porcine hydrogel (FIG. 9C-D). The ability of a scaffolding material to promote cell proliferation may be a positive indication for its biocompatibility. As shown, similar proliferation was observed after 96 h in both human and porcine hydrogels (FIG. 9E). Overall, the combined results of cell adhesion, migration and proliferation indicate the efficiency of the decellularization process and efficient removal of the remaining traces of toxic molecules.

### Example 2

### Materials and Methods

Culturing undifferentiated iPSCs: iPSCs were generated from peripheral blood mononuclear cells (PBMC). The undifferentiated cells were cultivated and expended on tissue culture plates, pre-coated with 1□g/cm² LamininMX (Biolamina), and were cultured at 37° C. with 5% CO₂. Undifferentiated iPSCs were maintained in NutriStem® (Biological Industries) medium. Medium was replaced daily and cells were passaged twice weekly using CTS™ TrypLE™ Select Enzyme (Thermo).

Spinal cord motor neuron transplant generation and differentiation:

Dissociated iPSCs cells were mixed with ˜0.5-1% omentum-based hydrogel (prepared as described in Example 1) at a volumetric ratio of 1:2-1:12. Droplets of 1.0-2.4 μL were generated using an automated dispensing device. The particles were crosslinked at 37° C., 20 minutes after which culture medium was added. Undifferentiated cells were cultured in NutriStem that was replaced daily, for 72 hours. Cells were differentiated as previously described (R. Edri et al., Advanced materials 31, 1803895 (2019). Briefly, after achieving ˜90% confluence, medium was changed to Knockout/DMEM, supplemented with 15% Knockout Serum, 0.5% 1-glutamine, 1% non-essential amino acids (Invitrogen), 10 μM B-mercaptoethanol, 10 mM SB-431542 (Tocris), 1 μM LDN-193189 (Tocris), and 3 μM CHIR-99021 and was gradually changed every 3 days to DMEM/F12 supplemented with N2 (Day 3 was ¾ Knockout/DMED and ¼ DMEM/F12 with N2 supplemented for the F12 portion only, day 6 was changed as ½ ½). On days 4 and 6, the motor neuron medium was supplemented with 1 μM retinoic acid and 1 μM purmorphamine (Tocris). On day 8, DMEM F/12 supplemented with N2, 30 ng/mL sonic hedgehog (R&D) and 1 μM retinoic acid was added to the cells (⅓ of the final volume, without changing medium). After day 10, the medium was changed to DMEM/F12 supplemented with N2, 5 μg/mL BDNF (R&D), 200 μM ascorbic acid (Sigma), 1 μM purmorphamine (Tocris) and 1 μM retinoic acid. From day 15, 5 μM DAPT (Tocris) was also added, and purmorphamine concentration was decreased to 500 nM. Medium was changed every 3 days up to day 30.

Immunostaining: Cellular particles were fixed in 4% paraformaldehyde (Sigma-Aldrich) for 24 h followed by incubation in 15% sucrose (Sigma-Aldrich) for 2 hours and preservation in 30% sucrose at 4° C. Cryopreserved particles were embedded in OCT and cryo-sectioned to 20 μm thick slices. Sections were placed on slides and kept at −80° C. until staining. Slides to be stained were fixed with 4% formaldehyde (Sigma-Aldrich), permeabilized with 0.1% (v/v) triton X-100, blocked with PBS, 4% bovine serum albumin (BSA), 5% goat serum (GS) and stained with the indicated primary antibodies followed by secondary antibodies. Sections were imaged and analyzed using EVOS™ M5000 Imaging system (ThermoFisher Scientific, USA).

Neurite outgrowth assay: For neurite outgrowth assay, particles at day 26 of differentiation were placed on 15 mg/mL Geltrex™ (Gibco, ThermoFisher)-coated plates. The constructs were cultured for 3 days before fixation in 4% formaldehyde and imaging using Zeiss Primovert inverted cell culture microscope. Following light microscopy imaging, the constructs were fixed in 4% formaldehyde for 20 min and stained for TUJI and were imaged and analyzed using EVOS™ M5000 Imaging system (ThermoFisher Scientific, USA).

Flow cytometry: For flow cytometry analysis, cells were isolated from particles by incubation with 0.25% trypsin-EDTA (Gibco, ThermoFisher) for 20 minutes at 37° C. followed by mechanical trituration. Trypsin-EDTA was neutralized by DMEM/F-12 (Biological Industries) supplemented with 20% FBS (Biological Industries). Medium containing cells and disintegrated particles was sieved through 70 μm cell strainer. Cells that passed the strainer were centrifuged (120 g, 5 min), re-suspended in DMEM/F-12 and kept on ice.

For membrane proteins, cells were stained with conjugated antibody or isotype control for 30 min at RT. For intracellular proteins, cells were fixed and permeabilized with transcription factor staining buffer set (Miltenyi Biotec) and incubated with conjugated antibody or isotype control for 30 min on ice. Cells were analyzed and data analysis was performed using CytoFlex V2-B4-R2 flow cytometer (Beckman Coulter, USA). Positive populations were gated according to unstained cells and appropriate isotype control. At least 2 biological replicates were analyzed.

NeuroCard: For evaluation of gene expression at different time point of particle maturation a designated plate was assembled to identify expression of genes from different types of cells, including stem cells, neural progenitor cells, astrocytes, glia, Schwann, oligodendrocytes, mature neurons, motor neurons, sensory neurons, glutamatergic/GABAergic/dopaminergic/cholinergic neurons, mesoderm and endoderm. Total RNA was extracted from particles at day 0, day 14 and day 21 using PureLink™ RNA Mini Kit (Invitrogen) and cDNA synthesized using iScript Advanced cDNA Synthesis Kit for RT-qPCR (Bio-Rad). Quantitative real-time PCR was performed using QuantStudio™ 5 Real-Time PCR System with SsoAdvanced Universal SYBR Green Super-mix. The expression of each tested gene at day 14 or 21 was compared to its expression at day 0.

### Results

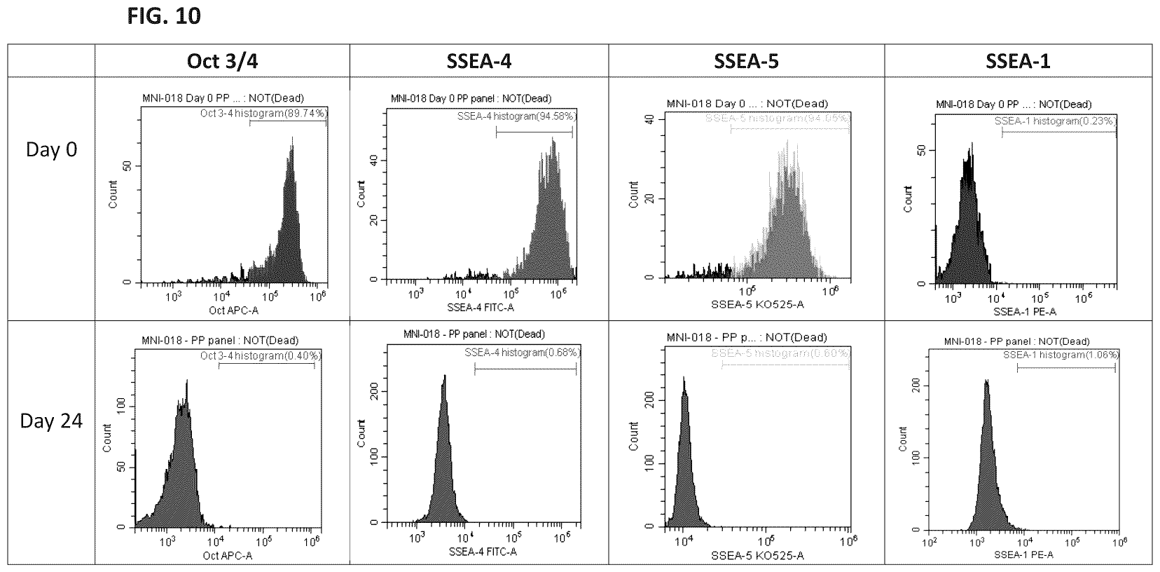

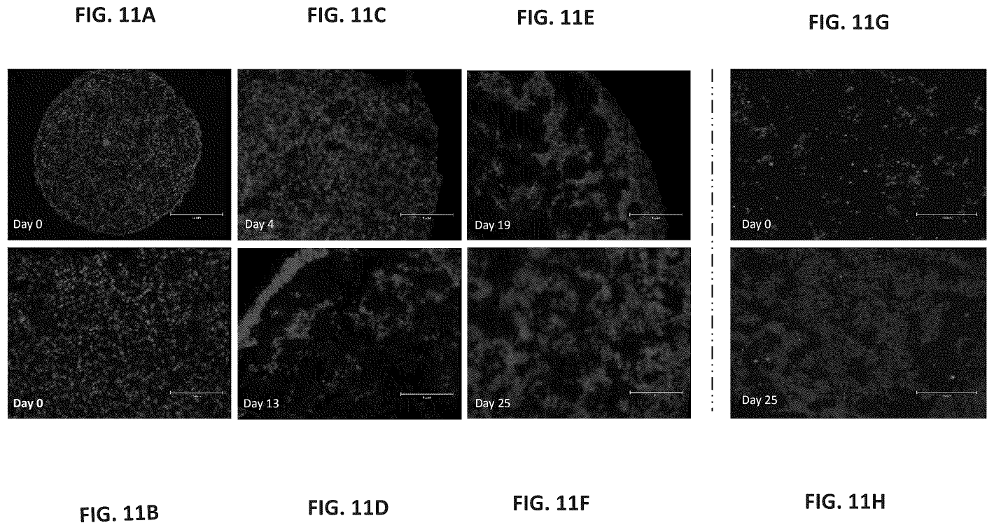

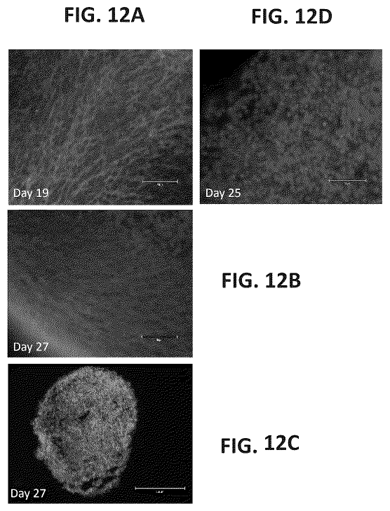

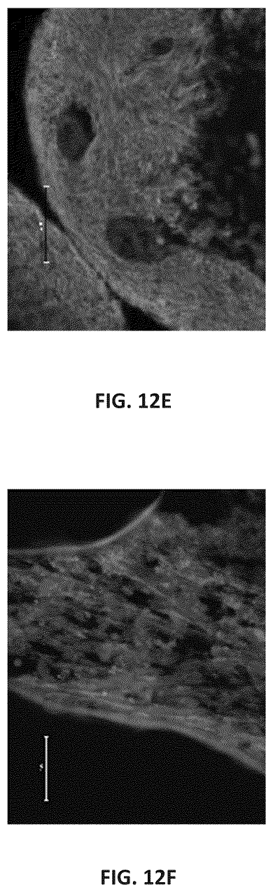

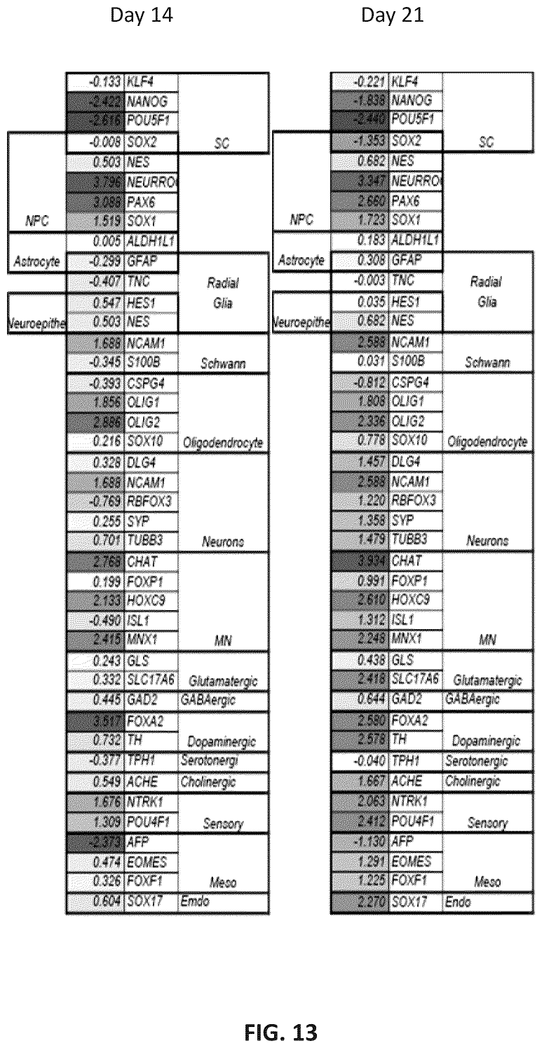

Neural transplants were generated, as described in the method section. As presented (FIG. 10), at day 0 (prior to differentiation) the cells present pluripotent nature as tested by flow cytometry (SSEA5, SSEA4, OCT4 and SSEA1). At day 24, no pluripotent marker is apparent as expected, due to exposure to the differentiation protocol. Furthermore, immunofluorescent staining of OCT4 at day 0 presents a homogeneous distribution of the pluripotent cells within the hydrogel (FIGS. 11A-B), on day 4 of differentiation low expression of OCT3/4 is observed (FIG. 11C) and at days 13, 19 and 25 (FIGS. 11D, E and F) no expression of OCT3/4 is seen. Furthermore, Ki67 staining indicating cell proliferation which is associated with pluripotent cell is apparent at day 0 and significantly reduces towards the end of differentiation (FIGS. 11G and H). Then, to mimic the physiological process of neurogenesis, the iPSCs particles were subjected to a 30 day differentiation protocol within the 3D microenvironment. Following differentiation, the cells formed a high-density 3D network within the entire particle (FIGS. 12A-F), expressing general early and late neuronal markers, such as TUJI (FIGS. 12 A, B and C) and NeuN (FIG. 12D), as well as the specific late motor neuron marker vesicular acetylcholine transporter (VAChT) protein (FIG. 12F) and HB9 (FIG. 12E), indicating the maturation of neural tissue. Moreover, gene expression at 2 different time points along the differentiation process (day 14 and day 21) revealed downregulation of pluripotency-associated genes, and upregulation of neuronal and specifically spinal cord motor neuron genes (FIG. 13). As shown, on day 14 there is a downregulation of pluripotent genes which is preserved at day 21. In parallel, upregulation of neuronal progenitor genes is apparent. In addition, at day 14 there is upregulation of glutamatergic, dopaminergic, cholinergic neurons, oligodendrocytes, motor neurons and sensory neurons which is elevated at day 21. No change is observed in astrocytes, glia or Schwann cells markers.

To assess the ability of the neural transplants to interact with their surrounding environment, isolated particles were seeded on a thin layer of Geltrex and neurite outgrowth was demonstrated (FIG. 14A). Furthermore, when several transplants were positioned at a distance of ˜1 mm from each other, a branched network was formed between them within 3 days (FIG. 14B). It was also presented that the neurites were positive when stained for TUJI (FIG. 14C) which further indicates that the branched network of neurite originated from the generated neural transplants. This interaction between several transplants and between transplants and the surrounding tissue is highly relevant to promote integration of the transplant and regeneration of the injured tissue.

### Example 3

### Materials and Methods

Decellularization of human omental tissue: Briefly, major blood vessels were manually removed from the tissue and the remaining tissue was chopped into 2-5 mm pieces with a scalpel. Samples were washed several times in PBS, before being agitated for 1 h in a hypotonic solution of 10 mM Tris 5 mM ethylenediaminetetraacetic acid (EDTA) at room temperature (RT). Then, in fresh hypotonic solution, the tissue was subjected to three cycles of freezing (−80° C.) and thawing (37° C.). After the last freeze-thaw cycle the tissue was gradually dehydrated by washing it once with 70% ethanol for 15 min and three times in 100% ethanol for 15 min each. Polar lipids of the tissue were then extracted by three 15 min washes of 100% acetone and a-polar lipids were extracted by four incubations in a 60:40 hexane: acetone solution (three incubations of 1.5 h and one incubation of 16 h). Then, the remaining tissue was gradually rehydrated and subjected to TrypLE (Thermo, cat #A1285901) degradation for 1.5 h at 37° C. The tissue was thoroughly washed with phosphate buffered saline (PBS) and with 50 mM Tris 1 mM MgCl₂. Afterwards, the tissue was gently agitated in a nucleic acid degradation solution of 50 mM Tris 1 mM MgCl₂ and 60 U/mL Denarase (c-Lecta) endonuclease for 20 h at 37° C. Finally, the tissue washed once with PBS and six times with sterile double distilled water (DDW). The decellularized tissue was stored frozen (−20° C.).

Preparation of solubilized human omentum dECM: The decellularized

omentum was cryo-milled and homogenized. Homogenized samples were enzymatically digested by adding a solution of pepsin 2000 FIP-U/g EMPROVE® ESSENTIAL (Merck) in 30 mM HCl (10 mg of pepsin enzyme per 1 ml DDW). After 1 hour, dry content was measured by moisture analyzer and a desired final Hydrogel concentration of the hydrogel was adjusted to 0.75% using 10 mg/ml pepsin solution. The dECM was digested for 48 h at RT under constant stirring, until the liquid was homogenous with no visible particles. Subsequently, the salt concentration was adjusted using PBS×10 and the pH was raised to 6.8-7.4 using 1 M NaOH. Raising the pH terminates pepsin activity (the enzyme is deactivated above pH 6).

Gelation kinetics: Gelation kinetics were evaluated spectrophotometrically. Briefly, 100 μL of hydrogels at 4° C. were transferred to 96-well plate in triplicates. Absorbance at 405 nm was measured every 30 s for 1 h using an Epoch 2 Microplate Spectrophotometer (BioTek), preheated to 37° C. OD values were then plotted over time and a Sigmoidal, 4PL curve was fitted over the results using GraphPad Prizm software. T50 (half time to gelation) and Span values of the curve were calculated automatically by the software, as well as the area under the curve (AUC). The T50 value provides an indication of the incubation time required for sufficient gelation during transplant creation. The Span and AUC parameters are indicative of the hydrogel quality—too low values point to poor gelation.

Mock transplant assessment: An important hydrogel characteristic is its texture, which can be assessed using the Mock transplant (MT) assay. If a manufactured hydrogel is insufficiently homogenous, it might form transplants of inconsistent sizes. A hydrogel is deemed to be of good quality when it can be used to produce droplets of comparable sizes that have well-defined outlines and smooth texture. Briefly, 50 droplets of 1 μl human-derived hydrogel were pipetted using an E3X dispenser onto a Petri dish and incubated at 37° C. to allow cross-linking and solidification. Then, 10 random MT were examined under a microscope to assess the following attributes:

MT diameter & distribution—measured by image analysis; average±SEM calculated from measured MT diameter.

MT outline—assessed visually and compared to a scale.

MT homogeneity—assessed visually and compared to a scale.

Denarase residues measurement: Residual Denarase levels in the hydrogel were measured using the DENERASE ELISA kit (C-Lecta) according to the manufacturer's instructions. Briefly, hydrogel samples were diluted in sample buffer and incubated in a pre-coated plate to allow antigen binding. Then, after washing of unbound components, detector antibody and enzyme conjugate were successively added and incubated in the well, followed by an additional incubation with substrate solution resulting in color development. OD was then measured and Denerase concentration was calculated from a standard curve.

DNA level measurement: Residual DNA extraction from the hydrogel was performed using the DNeasy kit (Qiagen). Samples were first lysed using proteinase K. Then, buffering conditions were adjusted to provide optimal DNA binding, and the lysate was loaded onto the DNeasy Mini spin column. During centrifugation, DNA was selectively bound to the DNeasy silica-based membrane, as contaminants passed through. The remaining contaminants and enzyme inhibitors were washed, and DNA was then eluted from the membrane using elution buffer. Quantification of the extracted DNA was performed by using the QuantiFluor® ONE dsDNA System—a highly selective fluorescent dsDNA-binding dye (504 nmEx/531 nmEm) prepared in an “add-and-read” format. In this case, extracted DNA samples were assayed against a standard curve of genomic DNA.

Proteomic LC-MS/MS analysis: Analysis was performed by Smoler Proteomics Center (Technion). Briefly, samples of human and porcine 100 μl solidified hydrogel following washing were digested with trypsin, analyzed by LC-MS/MS on Q-Exactive HF (Thermo) and identified by Discoverer software against either the Sus scrofa or the human proteome from the Uniprot database, and a decoy database (in order to determine the false discovery rate). All the identified peptides were filtered with high confidence, and the identified proteins with a minimum of 2 peptides. Peptide percentage out of the sample was calculated by IBAQ.

### Results

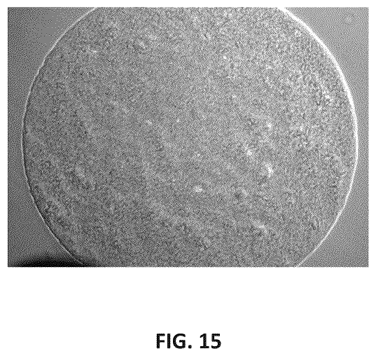

Mock transplant assessment: Hydrogel sample was assayed and droplets of similar diameters (1.74 mm, CV 2.6%) were measured. As illustrated in FIG. 15, the droplets had well-defined outlines (grade A) and a highly smooth and homogeneous texture (grade A).

Denarase residue measurement: In all tested hydrogels, Denarase levels were below quantification level (<46.875 pg/ml).

DNA level measurement: Following DNA removal with Denerase during the decellularization procedure, residual DNA was quantified in five hydrogel samples and was found to be 20±6.35 ng/mg dry weight.

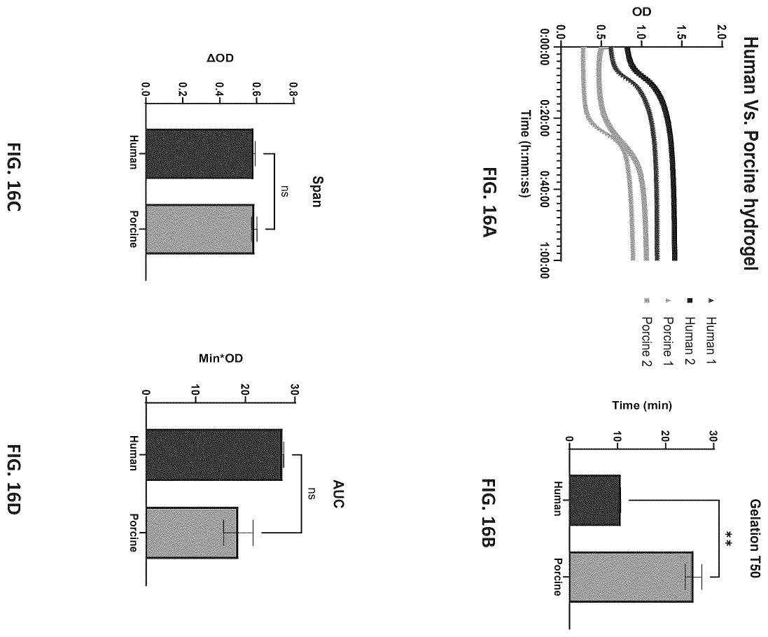

Gelation kinetics: The results are illustrated in FIGS. 16A-D. Gelation parameters calculated from the obtained graphs revealed different gelation kinetics. While both groups (human and porcine) had similar span and AUC values, faster gelation was obtained with human-based hydrogels compared to porcine-based hydrogels.

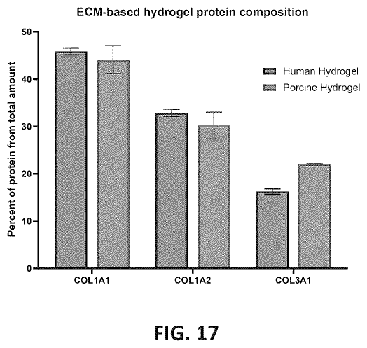

Proteomic LC-MS/MS analysis of hydrogel: As presented in FIG. 17, it is apparent that more than 95% of the proteins within both human and porcine hydrogel are collagens. It is notable that within the hydrogel the amount of collagen 3A1 is significantly lower in the human hydrogel as compared to the porcine hydrogel. It is expected that this difference results in the human hydrogel having different mechanical, chemical or structural properties than the porcine based hydrogel.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of generating transplantable engineered tissue comprising combining a liquid decellularized human omentum composition with cells.

2. The method of claim 1, wherein the cells comprise pluripotent stem cells.

3. The method of claim 2, wherein the pluripotent stem cells are induced pluripotent stem cells.

4. The method of claim 3, wherein the induced pluripotent stem cells are derived from peripheral blood mononuclear cells.

5. The method of claim 4, wherein the cells are differentiated into mature neural cells or neural progenitor cells within the liquid composition.

6. The method of claim 1, wherein the liquid composition of decellularized human omentum is an extracellular matrix (ECM) hydrogel, which solidifies upon heating to a temperature of above 30° C.

7. The method of claim 6, wherein the ECM hydrogel is prepared by enzymatic digestion of decellularized human omentuml.

8. The method of claim 7, wherein enzymatic digestion of decellularized human omentum comprises contacting decellularized human omentum with pepsin, trypsin, pancreatin, or any derivatives thereof.

9. The method of claim 1, further comprising dispensing droplets of the liquid composition of ECM hydrogel and cells onto a solid surface.

10. The method of claim 9, wherein the droplets are subjected to conditions that promote solidification.

11. The method of claim 10, wherein further solidified particles are cultured under conditions that promote cellular differentiation.

12. The method of claim 10, wherein the formed transplantable engineered tissue is in the form of particles having a diameter between 400 microns and 3 millimeters.

13. The method of claim 1, wherein the decellularized omentum is autologous to the subject.

14. A method of regenerating tissue in a subject in need thereof, comprising:
administering to the subject a therapeutically effective amount of an engineered tissue produced by the method of claim 1, wherein the engineered tissue promotes tissue regeneration and functional recovery at a site of injury or degeneration.

15. The method of claim 14, wherein the injury or degeneration is neuronal injury or neurodegenerative disease, thereby treating the neuronal injury or neurodegenerative disease.

16. The method of claim 15, wherein the neuronal injury or neurodegenerative disease comprises spinal cord injury, stroke or traumatic brain injury.

17. The method of claim 14, wherein said omentum and cells are autologous to the subject.
